(19) United States
(12) Patent Application Publication
(10) Pub. No.: US 2025/0066292 A1
(43) Pub. Date: Feb. 27, 2025

(54) CYSTEAMINE AND/OR CYSTAMINE PRODRUGS

(71) Applicant: The Regents of the University of California, Oakland, CA (US)

(72) Inventors: Ranjan Dohil, San Diego, CA (US); Carlo Ballatore, San Diego, CA (US)

(21) Appl. No.: 18/724,564

(22) PCT Filed: Dec. 28, 2022
(86) PCT No.: PCT/US2022/054144

§ 371 Date: Jun. 26, 2024

Related U.S. Application Data
(60) Provisional application No. 63/294,335, filed on Dec. 28, 2021.

(51) Int. Cl. C07C 327/30; A61K 31/223; A61K 31/265; A61P 1/16; C07C 323/58

(52) U.S. Cl. CPC C07C 327/30; A61K 31/223; A61K 31/265; A61P 1/16; C07C 323/58; C07B 2200/05

(57) ABSTRACT

The disclosure provides for cysteamine prodrugs, pharmaceutical compositions made thereof, and methods thereof including the treatment of any disease or disorder in a subject that can benefit from one or more of the bioprotective effects of cysteamine, including but not limited to, binding of cystine, reducing oxidative stress, increasing adiponectin levels and/or increasing brain-derived neurotrophic factors. Examples of such disease and disorders, include but are not limited to, cystinosis, and fatty liver diseases including non-alcoholic steatohepatitis (NASH).

### CROSS REFERENCE TO RELATED APPLICATIONS

This application claims priority of U.S. Provisional Application No. 63/294,335, filed Dec. 28, 2021, the disclosures of which are incorporated herein by reference in its entirety.

### FIELD OF THE INVENTION

Disclosed herein are cysteamine prodrugs, pharmaceutical compositions made thereof, and methods thereof including the treatment of any disease or disorder in a subject that can benefit from one or more of the bioprotective effects of cysteamine, including but not limited to, binding of cystine, reducing oxidative stress, increasing adiponectin levels and/or increasing brain-derived neurotrophic factors. Examples of such disease and disorders, include but are not limited to, cystinosis, and fatty liver diseases including non-alcoholic steatohepatitis (NASH).

### BACKGROUND

Cysteine is a commonly found amino-acid which exists in the human body mainly in its oxidized form cystine. Cysteine is essential for the production of the potent anti-oxidant glutathione. Unfortunately, the cellular uptake of cysteine (mostly as cystine) is rate limited. Many conditions are associated with oxidative stress including non-alcoholic steatohepatitis (NASH) and some neurodegenerative disorders. Cystamine is the dimeric oxidized form of cysteamine. Cysteamine has been shown to reverse hepatic inflammation associated with NASH. Abnormal intralysosomal cystine deposition occurs in nephropathic cystinosis resulting in organ failure. Cysteamine will reduce the cystine accumulation and therefore improve the prognosis in these patients. However, this drug is associated with side effects such as gastrointestinal symptoms, halitosis and body odor and are more likely to occur when plasma levels (Cₘₐₓ) of cysteamine are high.

### SUMMARY

Cysteamine is a well-recognized treatment for nephropathic cystinosis, but is associated with side-effects. It is commercially available as cysteamine bitartrate and given as a q6 h immediate-release formulation (Cystagon) and a q12 h delayed-release formulation (Procysbi®). Both of these formulations are associate with adverse effects, although less so with Procysbi®. The latter requires large numbers of pills to be ingested daily. There are no accepted and commercially available drug therapies for NASH. Cystamine is not commercially available for the treatment of any medical disorder.

The compounds of the disclosure, in some embodiments, are prodrugs of cysteamine that is bound to cysteine. The cysteamine-cysteine may be considered “mutual prodrugs” such that each moiety (cysteamine or cysteine) have biological activity. Cysteamine is a recognized anti-oxidant, although not commercially approved for conditions associated with increased oxidative stress. Adverse effects of cysteamine are associated with a higher Cₘₐₓ (i.e., higher plasma concertation). The compounds of the disclosure deliver cysteamine and cysteine intracellularly where the prodrug is reduced to cysteamine inside the cell. Cysteamine is an anti-oxidant and cysteine is essential for the production of the body's most important anti-oxidant glutathione. Intracellular uptake of cystine is rate limited and this therefore would normally control the availability of cysteine. The compounds of the disclosure enter the cell through passive diffusion and/or different transport pathways including the passage of sulfides, disulfides and mixed disulfides. Where cystamine is present each cystamine compound yields two molecules of cysteamine, a relatively small dose of the compound can be delivered in comparison to cysteamine bitartrate. Certain compounds described in the disclosure, in addition to delivering cysteamine, upon prodrug activation, a molecule of cysteine or related compound would be delivered intracellularly, which would facilitate the production of glutathione.

In a particular embodiment, the disclosure provides for a compound having the structure of Formula I:

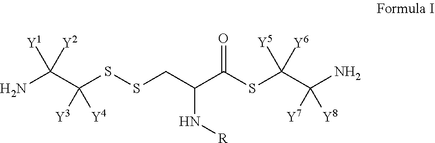

or a pharmaceutically acceptable salt or solvate thereof, wherein: Y¹-Y⁸ are each independently selected from H or D; and R is selected from H or an acetyl group. In another embodiment, at least one of Y¹-Y⁸ independently has deuterium enrichment of no less than about 10%. In yet another embodiment, at least one of Y¹-Y⁸ independently has deuterium enrichment of no less than about 50%. In a further embodiment, at least one of Y¹-Y⁸ independently has deuterium enrichment of no less than about 90%. In yet a further embodiment, at least one of Y¹-Y⁸ independently has deuterium enrichment of no less than about 98%. In a certain embodiment, the compound has a structural formula of Formula I(a):

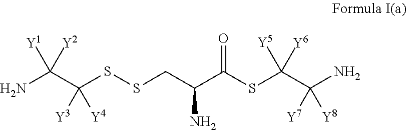

or a pharmaceutically acceptable salt or solvate thereof, wherein: Y¹-Y⁸ are each independently selected from H or D. In another embodiment, the compound has a structural formula selected from:

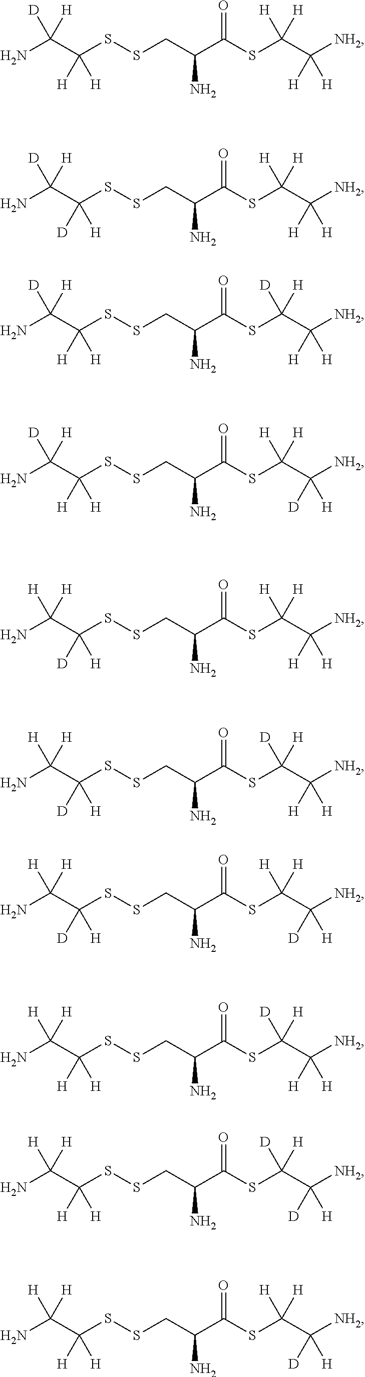

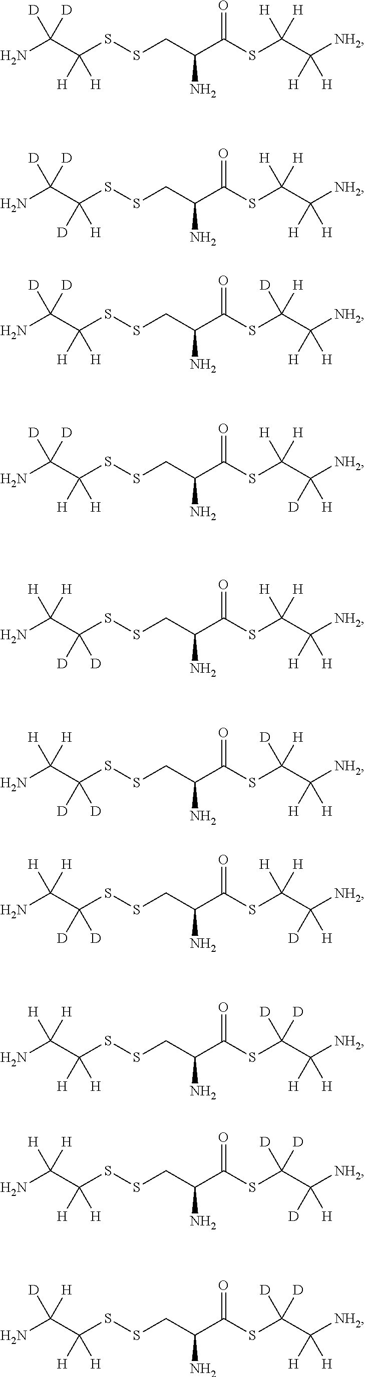

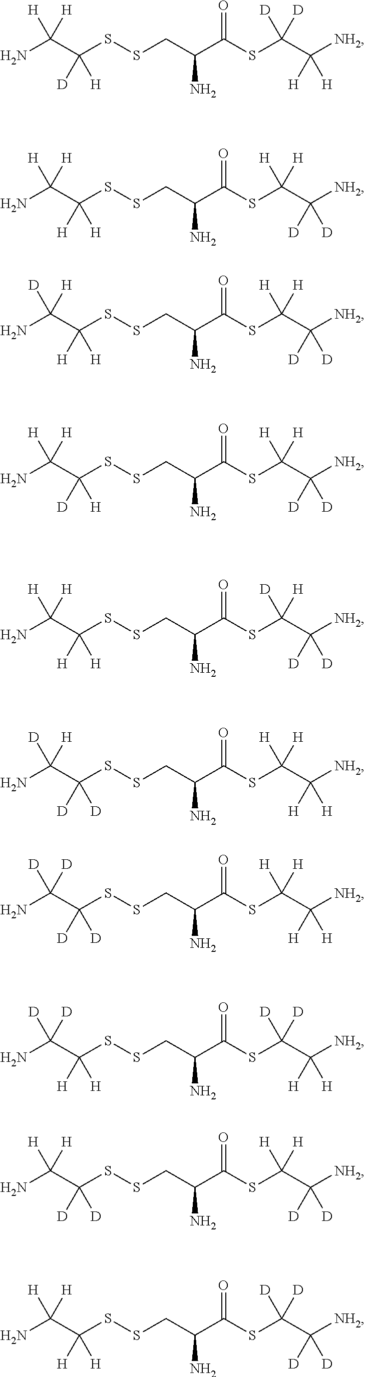

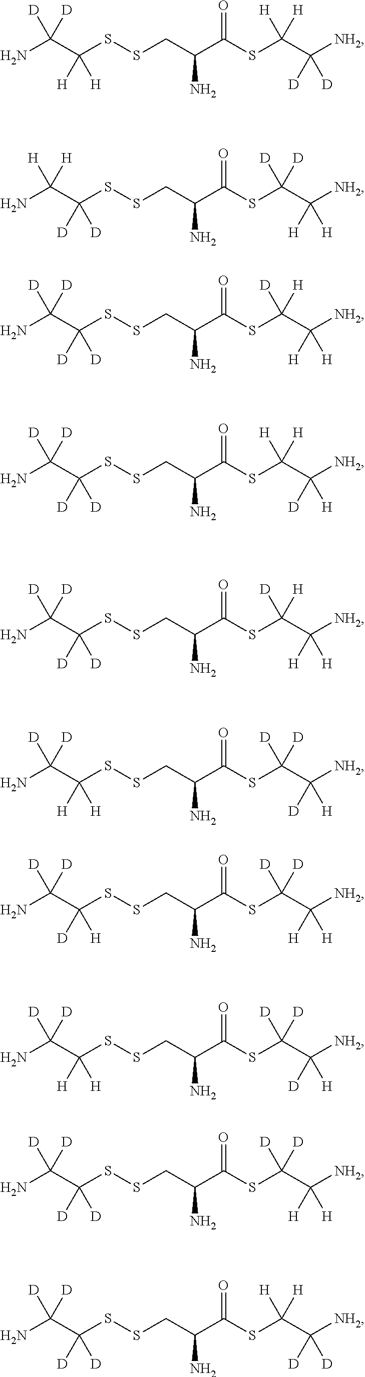

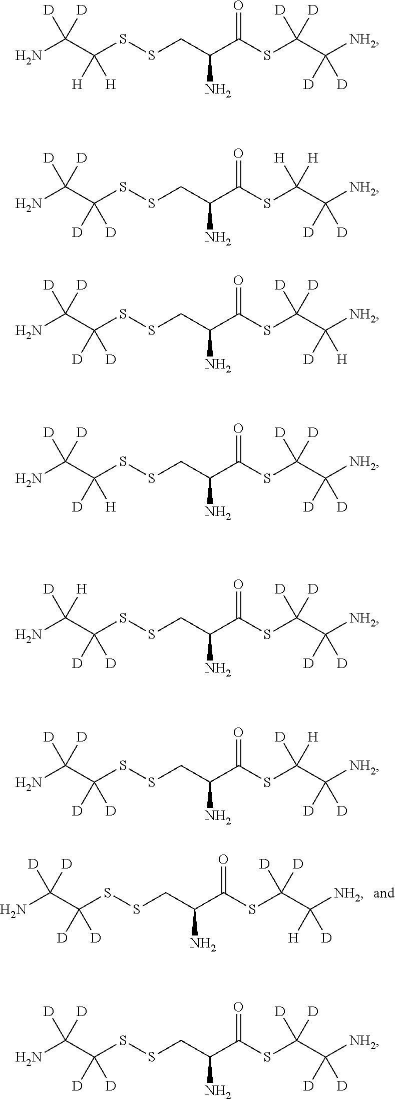

or a pharmaceutically acceptable salt or solvate of any one of the foregoing. In a further embodiment, the compound has a structure of:

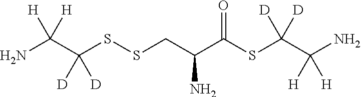 [SMILES: [2H]C([2H])(CN)SSC[C@H](N)C(=O)SC([2H])([2H])CN]

or a pharmaceutically acceptable salt, solvate, or prodrug thereof. In yet a further embodiment, the pharmaceutically acceptable salt has the structure of Formula I(b):

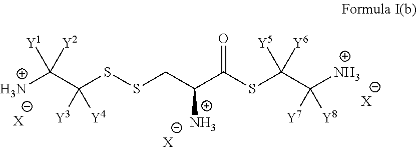

wherein, Y¹-Y⁸ are each independently selected from H or D; and X is a pharmaceutically acceptable counter ion. In yet a further embodiment, the pharmaceutically acceptable counter ion is a bitartrate ion or chloride ion. In another embodiment, the compound has the structure of

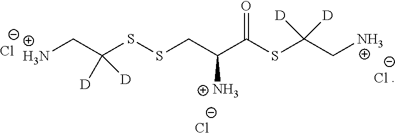 [SMILES: [Cl-] ; [2H]C([2H])(C[NH3+])SSC[C@H]([NH3+])C(=O)SC([2H])([2H])C[NH3+] ; [Cl-] ; [Cl-]]

In yet another embodiment, the compound has the structure of Formula I(c):

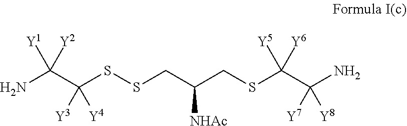

or a pharmaceutically acceptable salt or solvate thereof, wherein: Y¹-Y⁸ are each independently selected from H or D; and Ac refers to an acetyl group. In a further embodiment, the compound has a structural formula selected from:

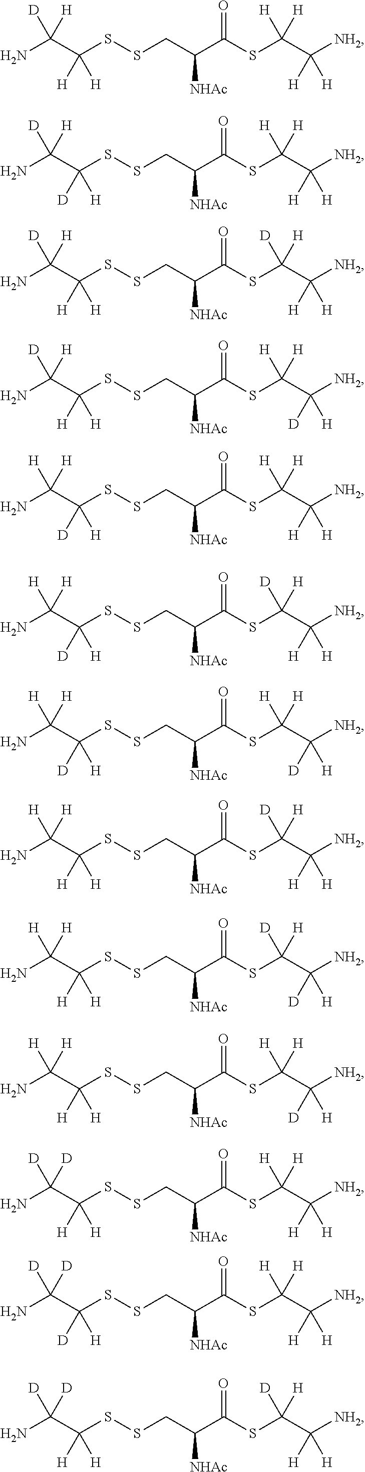

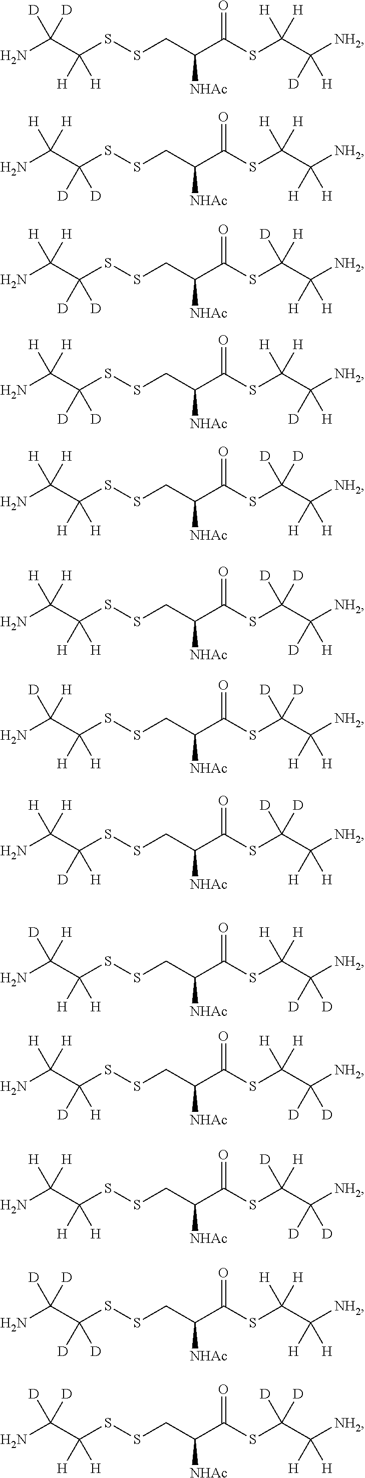

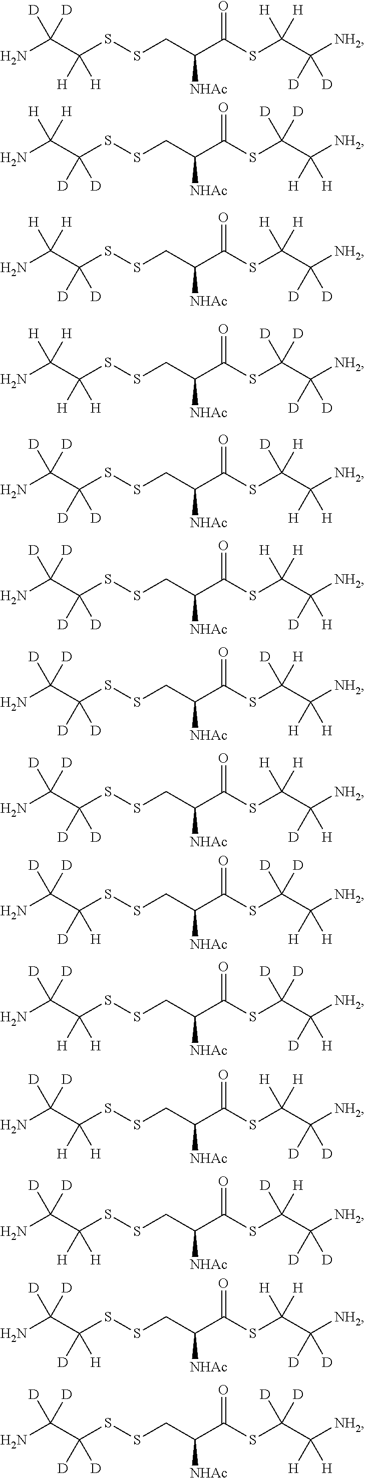

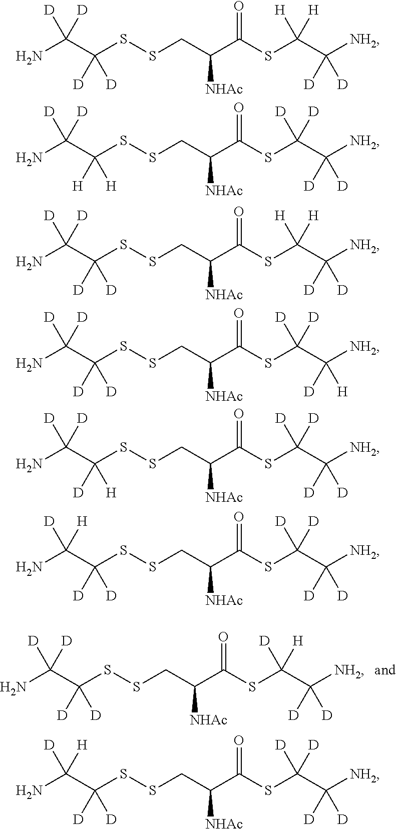

or a pharmaceutically acceptable salt or prodrug thereof. In a certain embodiment, the pharmaceutically acceptable salt has the structure of Formula I(d):

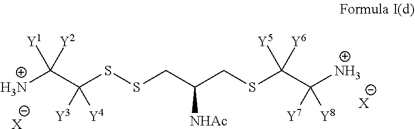

wherein, Y¹-Y³ are each independently selected from H or D; Ac is an acetyl group; and X is a pharmaceutically acceptable counter ion. In another embodiment, the pharmaceutically acceptable counter ion is a bitartrate ion or chloride ion. In yet another embodiment, the compound has the structure of:

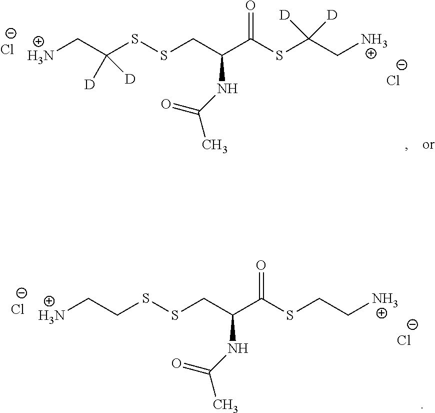 [SMILES: [Cl-] ; [2H]C([2H])(C[NH3+])SSC[C@H](NC(C)=O)C(=O)SC([2H])([2H])C[NH3+] ; [Cl-] ; [Cl-] ; CC(=O)N[C@@H](CSSCC[NH3+])C(=O)SCC[NH3+] ; [Cl-]]

In a further embodiment, the compound has a structural formula of Formula III(a):

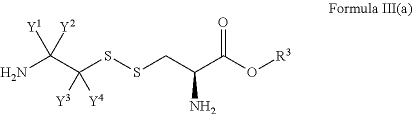

or a pharmaceutically acceptable salt or solvate thereof, wherein:
Y¹-Y⁴ are each independently selected from H or D; and
R³ is selected from a (C₁-C₆)alkyl. In a further embodiment, the compound has a structural formula selected from:

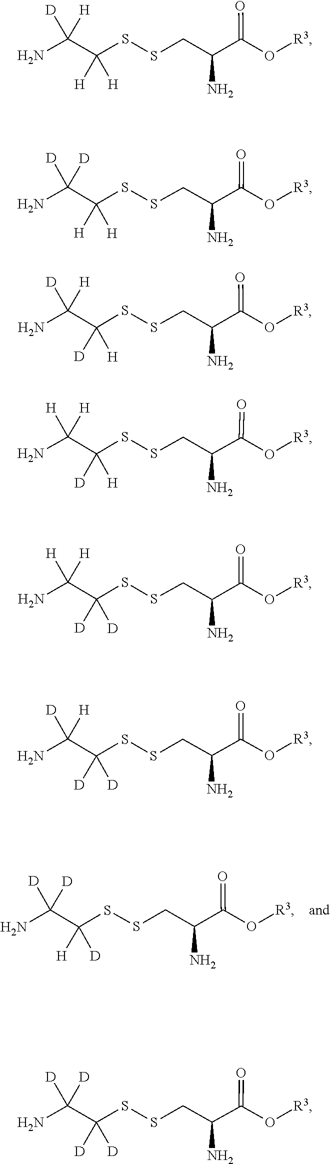

or a pharmaceutically acceptable salt or solvate of any one of the foregoing, wherein R³ is a (C₁-C₆)alkyl. In another embodiment, the pharmaceutically acceptable salt has the structure of Formula III(b):

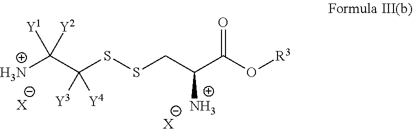

wherein, Y¹-Y⁴ are each independently selected from H or D;
R³ is a (C₁-C₆)alkyl; and X is a pharmaceutically acceptable counter ion. In a further embodiment, the compound has the structure of Formula III(c):

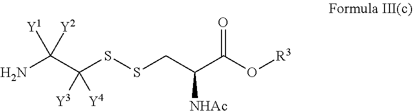

or a pharmaceutically acceptable salt or solvate thereof, wherein: Y¹-Y⁴ are each independently selected from H or D; and R³ is a (C₁-C₆)alkyl. In yet a further embodiment, the compound has a structural formula selected from:

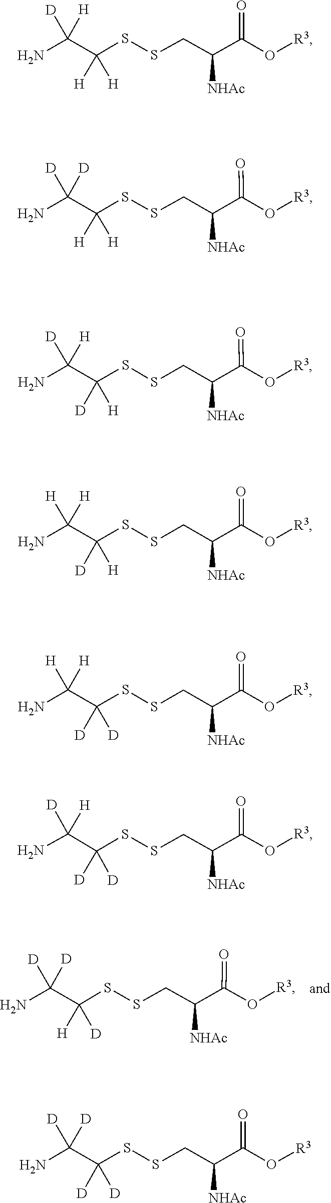

or a pharmaceutically acceptable salt or prodrug thereof, wherein R³ is a (C₁-C₆)alkyl. In another embodiment, the pharmaceutically acceptable salt has the structure of Formula III(d):

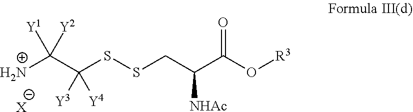

wherein, Y¹-Y⁴ are each independently selected from H or D;
R³ is a (C₁-C₆)alkyl; and X is a pharmaceutically acceptable counter ion. In a certain embodiment, the compound has the structure of Formula IV:

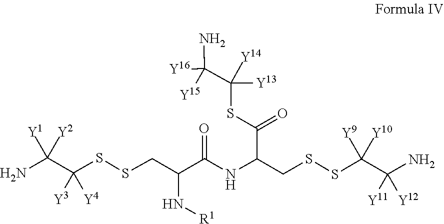

or a pharmaceutically acceptable salt or solvate thereof, wherein:
Y¹-Y⁴ and Y¹-Y¹⁶ are each independently selected from H or D; and
R¹ is selected from H or an acetyl group. In another embodiment, the compound has the structure of Formula V:

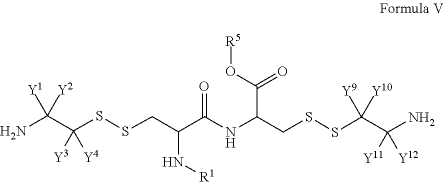

or a pharmaceutically acceptable salt or solvate thereof, wherein:
Y¹-Y⁴ and Y¹-Y¹⁶ are each independently selected from H or D;
R¹ is selected from H or an acetyl group; and
R⁵ is a (C₁-C₆)alkyl.

The disclosure also provides a compound of Formula VI:

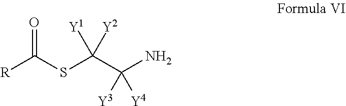

or a pharmaceutically acceptable salt or solvate thereof, wherein: Y¹-Y⁴ are each independently selected from H or D; and R is linear or branched aliphatic group (saturated or unsaturated) or aromatic (substituted or non-substituted) having from 1 to 20 carbon atoms. In another embodiment, at least one of Y¹-Y⁴ independently has deuterium enrichment of no less than about 10%. In yet another embodiment, at least one of Y¹-Y⁴ independently has deuterium enrichment of no less than about 50%. In a further embodiment, at least one of Y¹-Y⁴ independently has deuterium enrichment of no less than about 90%. In yet a further embodiment, at least one of Y¹-Y⁴ independently has deuterium enrichment of no less than about 98%. In one embodiment, the compound comprises Formula VI(a):

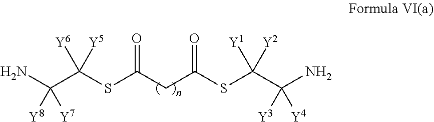

or a pharmaceutically acceptable salt or solvate thereof, wherein: Y¹-Y⁸ are each independently selected from H or D; and n is 2-6 (e.g., 2, 3, 4, 5, or 6). In another embodiment, at least one of Y¹-Y⁸ independently has deuterium enrichment of no less than about 10%. In yet another embodiment, at least one of Y¹-Y⁸ independently has deuterium enrichment of no less than about 50%. In a further embodiment, at least one of Y¹-Y⁸ independently has deuterium enrichment of no less than about 90%. In yet a further embodiment, at least one of Y¹-Y⁸ independently has deuterium enrichment of no less than about 98%. In still another embodiment, the compound of Formula VI are selected from:

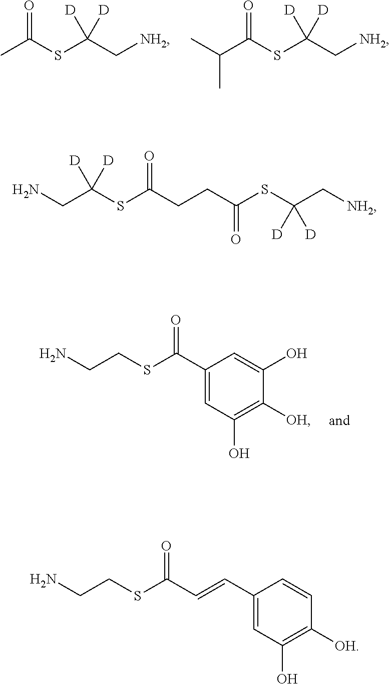

In a particular embodiment, the disclosure also provides a pharmaceutical composition comprising a compound disclosed herein and a pharmaceutically acceptable carrier, diluent, and/or binder. In a further embodiment, the pharmaceutical composition is formulated for oral delivery. In yet a further embodiment, the composition is the in the form of granules, tablet, capsule, or caplet. In another embodiment, the pharmaceutical composition is formulated for delayed release. In yet another embodiment, the pharmaceutical composition comprises an enteric coating.

In a certain embodiment, the disclosure further provides a method of treating a subject suffering from a disease or disorder selected from the group consisting of cystinosis, fatty liver disease, cirrhosis, an eosinophilic disease or disorder, and Huntington's disease, comprising administering to the subject a therapeutically effective amount of a compound disclosed herein or a pharmaceutical composition of the disclosure. In a further embodiment, the subject suffers from cystinosis. In yet a further embodiment, the disease or disorder is a fatty liver disease. In another embodiment, the fatty liver disease is selected from the group consisting of non-alcoholic fatty liver disease (NAFLD), non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy. In a certain embodiment, the fatty liver disease is non-alcoholic steatohepatitis (NASH). In another embodiment, the method further comprises measuring one or more markers of liver function selected from the group consisting of alanine aminotransferase (ALT), alkaline phosphatase (ALP), aspartate aminotransferase (AST), gamma-glutamyl transpeptidase (GGT) and triglycerides. In yet another embodiment, an ALT level of about 60-150 units/liter is indicative of fatty liver disease and wherein the compound improves ALT levels. In yet another embodiment, an ALP level of about 150-250 units/liter is indicative of fatty liver disease and wherein the compound improves ALP levels. In yet another embodiment, an AST level of about 40-100 units/liter is indicative of fatty liver disease and wherein the compound improves AST levels. In yet another embodiment, a GGT level of 50-100 units/liter is indicative of fatty liver disease and wherein the compound improves GGT levels. In one embodiment, the liver disease or disorder is selected from the group consisting of NAFLD with pediatric type 2 pattern, autoimmune hepatitis, primary sclerosing cholangitis, primary biliary cholangitis, chronic drug toxicity, biliary atresia, idiopathic neonatal hepatitis syndrome.

In a particular embodiment, the disclosure provides a method of reducing fibrosis or fat content or fat accumulation in the liver associated with non-alcoholic fatty liver disease (NAFLD) comprising administering a compound disclosed herein or a pharmaceutical composition of the disclosure. In a further embodiment, the NALFD comprises NASH.

### BRIEF DESCRIPTION OF THE DRAWINGS

FIG. 1 provide synthesis schemes to make exemplary compounds of the disclosure.

FIG. 2 provides the results of a cystine depletion study with constant compound exposure. As shown, the depletion kinetics were nearly identical between the tested experimental compound (BL-0856) and cystamine.

FIG. 3 presents the results of a washout experiment after a 3 h incubation with the tested experimental compound (BL-0856). The rate of cystine reaccumulation with the experimental compound was comparable to that of cystamine.

FIG. 4 presents the measured amounts of cysteamine or cystamine and the experimental compound (BL-0856) over time after being administered to cells. As expected, cystamine levels are higher in cystamine treated, yet some cystamine still exists in the compound treated cells. Cysteamine levels are nearly identical in both treatments, indicating that reduction of cysteamine from disulfide precursors is rapid in both.

FIG. 5 presents the measured amounts of glutathione (GSH), oxidized glutathione (GSSG) and the ratio of GSH/GSSG over time after cystamine or the experimental compound (BL-0856) are administered to cells. No obvious differences in glutathione between 2 drug forms. Of interest is that with both drugs, oxidized glutathione (GSSG) goes up at the first timepoint, and ratio of GSH/GSSG is lower throughout all timepoints in comparison to the 0-minute controls.

FIG. 6 presents the measured amounts of cysteamine over time after cystamine or the experimental compound (BL-0856) are administered to cells. As expected, cysteine is higher in the BL-0856 treated cells, indicating that cysteine is being released from the precursor form.

FIG. 7 shows intracellular D2-cysteamine levels following continual drug exposure (in media) to cystinotic fibroblasts.

FIG. 8 shows cystine levels after treatment of cystinotic fibroblasts with D4-cystamine, compound 0940 and 0948.

FIG. 9 shows time course of cysteamine production by D4-cystamine, compound 0940 and compound 0948.

FIG. 10A-E shows (A) depiction of the experimental study; (B) ratio of liver weight (LW) to body weight (BW) of mice in the study for each therapy group; (C) ALT activity for each drug group; (D) H&E and Sirius Red staining of liver section for each drug group and (E) gene expression of col1 for each group.

FIG. 11 show graphs of BW/LW and ALT activity for control, Drug 2 and Drug 4 groups.

FIG. 12A-B shows (A) staining of liver section for each drug group for markers of inflammation; (B) shows changes of inflammatory cytokines in each study group.

FIG. 13A-C shows (A) aSMA staining for each drug study group; (B) expression levels of inflammatory markers including matrix metalloproteases; and (C) aSMA protein staining levels.

FIG. 14 shows inflammatory marker measurements for prodrugs in NASH mouse models.

FIG. 15 shows fibrosis marker measurements for prodrugs in NASH mouse models.

FIG. 16 shows markers of tissue remodeling in NASH mouse models receiving prodrug therapy.

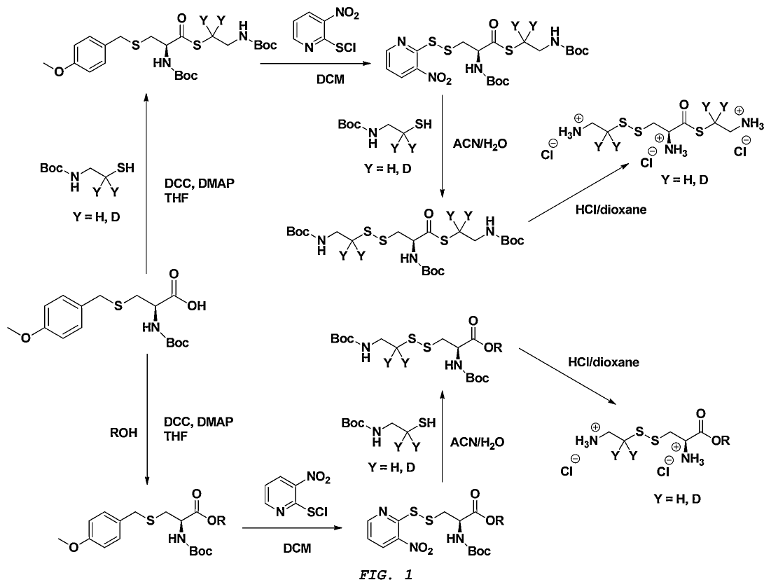

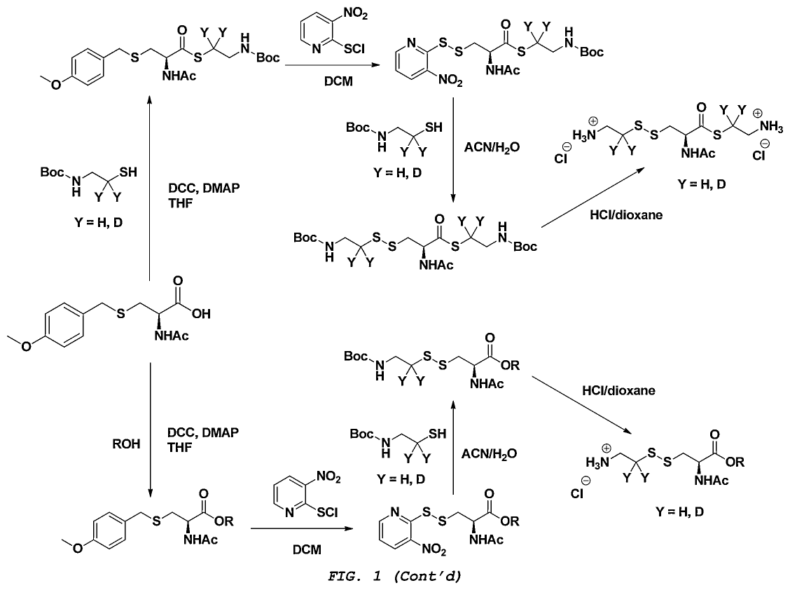

### DETAILED DESCRIPTION

As used herein and in the appended claims, the singular forms “a,” “an” and “the” include plural referents unless the context clearly dictates otherwise. Thus, for example, reference to “a compound” includes a plurality of such compounds and reference to “the subject” includes reference to one or more subjects and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

The term “about” as used herein can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The terms “active ingredient”, “active compound”, and “active Substance” refer to a compound, which is administered, alone or in combination with one or more pharmaceutically acceptable excipients or carriers, to a subject for treating, preventing, or ameliorating one or more symptoms of a disorder.

In representing a range of positions on a structure, the notation “from R^{x} to R^{xx}” or “R^{x}-R^{xx}” may be used, wherein X and XX represent numbers. Then unless otherwise specified, this notation is intended to include not only the numbers represented by X and XX themselves, but all the numbered positions that are bounded by X and XX. For example, “from R¹ to R⁴” or “R¹-R⁴” would, unless otherwise specified, be equivalent to R¹, R², R³, and R⁴.

The term “combination therapy’ means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials are about 0.0156%. The deuterium enrichment can be determined using conventional analytical methods known to one of ordinary skill in the art, including mass spectrometry and nuclear magnetic resonance spectroscopy.

The term “is/are deuterium”, when used to describe a given position in a molecule such as R—R or the symbol “D.” when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “disorder” as used herein is intended to be generally synonymous, and is used interchangeably with, the terms “disease”, “syndrome”, and “condition” (as in medical condition), in that all reflect an abnormal condition of the human or animal body or of one of its parts that impairs normal functioning, is typically manifested by distinguishing signs and Symptoms.

The terms “drug” and “therapeutic agent” refer to a compound, or a pharmaceutical composition thereof, which is administered to a subject for treating, preventing, or ameliorating one or more symptoms of a disease or disorder.

The term “non-deuterated” when used to describe a compound refers to a compound that has not been manufactured to increase the level of deuteration beyond what may naturally occur without the process of active deuteration. In some instances, a non-deuterated molecule lacks any deuterated atoms.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

The term “non-release controlling excipient” refers to an excipient whose primary function does not include modifying the duration or place of release of the active substance from a dosage form as compared with a conventional immediate release dosage form.

The term “pharmaceutically acceptable carrier, “pharmaceutically acceptable excipient”, “physiologically acceptable carrier”, or “physiologically acceptable excipient” refers to a pharmaceutically-acceptable material, composition, or vehicle, such as a liquid or Solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington. The Science and Practice of Pharmacy, 21st Edition; Lippincott Williams & Wilkins: Philadelphia, Pa., 2005, Handbook of Pharmaceutical Excipients, 5th Edition; Rowe et al., Eds. The Pharmaceutical Press and the American Pharmaceutical Association:2005; and Handbook of Pharmaceutical Additives, 3rd Edition; Ash and Ash Eds. Gower Publishing Company: 2007; Pharmaceutical Preformulation and Formulation, Gibson Ed., CRC Press LLC: Boca Raton, Fla., 2004).

The compounds disclosed herein can and do exist as therapeutically acceptable salts. The term “pharmaceutically acceptable salt”, as used herein, represents salts or Zwitterionic forms of the compounds disclosed herein which are therapeutically acceptable as defined herein. The salts can be prepared during the final isolation and purification of the compounds or separately by reacting the appropriate compound with a suitable acid or base. Therapeutically accept able salts include acid and basic addition salts. For a more complete discussion of the preparation and selection of salts, refer to “Handbook of Pharmaceutical Salts, Properties, and Use.” Stah and Wermuth, Ed. (Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-Sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, C—OXO-glutaric acid, glycolic acid, hippuric acid, hydrobromic acid, hydrochloric acid, hydroiodic acid, (+)-L-lactic acid, (t)-DL-lactic acid, lactobionic acid, lauric acid, maleic acid, (−)-L-malic acid, malonic acid, (+)-DL mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The terms “prevent”, “preventing”, and “prevention” refer to a method of delaying or precluding the onset of a disorder, and/or its attendant symptoms, barring a subject from acquiring a disorder or reducing a subject's risk of acquiring a disorder.

The term “prodrug” refers to a compound as disclosed herein that is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs. Roche Ed., APHA Acad. Pharm. Sci. 1977: “Bioreversible Carriers in Drug in Drug Design, Theory and Application.” Roche Ed., APHA Acad. Pharm. Sci. 1987: “Design of Prodrugs.” Bundgaard, Elsevier, 1985; Wang et al., Curr. Pharm. Design 1999, 5, 265-287: Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems.” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209: Browne, Clin. Neuropharmacol. 1997, 20, 1-12; Bundgaard, Arch. Pharm. Chem. 1979, 86, 1-39: Bundgaard, Controlled Drug Delivery 1987, 17, 179-96: Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877: Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94: Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The term “release controlling excipient” refers to an excipient whose primary function is to modify the duration or place of release of the active substance from a dosage form as compared with a conventional immediate release dosage form.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, Swine (e.g., pig, miniature pig), equine, canine, feline, and the like. The terms “subject’ and “patient” are used interchangeably herein in reference, for example, to a mammalian subject, such as a human patient.

The term “substantially” as used herein refers to a majority of, or mostly, as in at least about 51%, 60%, 70%, 80%, 90%, 95%, 96%, 97%, 98%, 99%, 99.5%, 99.9%, 99.99%, or at least about 99.999% or more.

The term “therapeutically acceptable” refers to those compounds (or salts, prodrugs, tautomers, Zwitterionic forms, etc.) which are suitable for use in contact with the tissues of patients without excessive toxicity, irritation, allergic response, immunogenicity, are commensurate with a reasonable benefit/risk ratio, and are effective for their intended use.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, Veterinarian, medical doctor, or clinician.

The terms “treat”, “treating”, and “treatment” are meant to include alleviating or abrogating a disorder or one or more of the symptoms associated with a disorder; or alleviating or eradicating the cause(s) of the disorder itself. As used herein, reference to “treatment of a disorder” is intended to include prevention.

Cysteamine is a small aminothiol molecule that is easily transported across cellular membranes. Cysteamine markedly reduces intralysosomal cysteine accumulation and is currently approved as a treatment for cystinosis. Cysteamine can increase the cellular thiol and free thiol tripeptide glutathione pool, and thus modulate reactive oxygen species (ROS) scavenging, and decreased lipoperoxidation and glutathione peroxidase activity. Furthermore, cysteamine also increases adiponectin levels.

Cysteamine is an attractive candidate for the treatment of fatty liver disease including NASH, as it reacts with cystine to produce cysteine, which can further be metabolized into glutathione, a potent endogenous antioxidant. Cysteamine is a precursor to the protein glutathione (GSH) precursor, and is currently FDA approved for use in the treatment of cystinosis, an intra-lysosomal cystine storage disorder. In cystinosis, cysteamine acts by converting cystine to cysteine and cysteine-cysteamine mixed disulfide which are then both able to leave the lysosome through the cysteine and lysine transporters respectively (Gahl et al., N Engl J Med 2002; 347(2):111-21). Within the cytosol the mixed disulfide can be reduced by its reaction with glutathione and the cysteine released can be used for further GSH syntheses. The synthesis of GSH from cysteine is catalyzed by two enzymes, gamma-glutamylcysteine synthetase and GSH synthetase. This pathway occurs in almost all cell types, with the liver being the major producer and exporter of GSH. The reduced cysteine-cysteamine mixed disulfide will also release cysteamine, which, in theory is then able to re-enter the lysosome, bind more cystine and repeat the process (Dohil et al., J Pediatr 2006; 148(6):764-9). In a recent study in children with cystinosis, enteral administration of cysteamine resulted in increased cysteamine absorption, which subsequently caused prolonged efficacy in the lowering of leukocyte cystine levels (Dohil et al., J Pediatr 2006; 148(6):764-9). This may have been due to “re-cycling” of cysteamine when adequate amounts of drug reached the lysosome. If cysteamine acts in this fashion, then GSH production may also be significantly enhanced.

Cysteamine is a potent gastric acid-secretagogue that has been used in laboratory animals to induce duodenal ulceration. Studies in humans and animals have shown that cysteamine-induced gastric acid hypersecretion is most likely mediated through hypergastrinemia. In previous studies performed in children with cystinosis who suffered regular upper gastrointestinal symptoms, a single oral dose of cysteamine (11-23 mg/kg) was shown to cause hypergastrinemia and a 2 to 3-fold rise in gastric acid-hypersecretion, and a 50% rise in serum gastrin levels. Symptoms suffered by these individuals included abdominal pain, heartburn, nausea, vomiting, and anorexia. U.S. patent application Ser. No. 11/990,869 and published International Publication No. WO 2007/089670, both claiming priority to U.S. Provisional Patent application No. 60/762,715, filed Jan. 26, 2006, (all of which are incorporated by reference herein in their entirety) showed that cysteamine induced hypergastrinemia arises, in part, as a local effect on the gastric antral-predominant G-cells in susceptible individuals. The data also suggest that this is also a systemic effect of gastrin release by cysteamine. Depending on the route of administration, plasma gastrin levels usually peak after intragastric delivery within 30 minutes whereas the plasma cysteamine levels peak later.

In addition, sulfhydryl (SH) compounds such as cysteamine, cystamine, and glutathione are among the most important and active intracellular antioxidants. Cysteamine protects animals against bone marrow and gastrointestinal radiation syndromes. The rationale for the importance of SH compounds is further supported by observations in mitotic cells. These are the most sensitive to radiation injury in terms of cell reproductive death and are noted to have the lowest level of SH compounds. Conversely, S-phase cells, which are the most resistant to radiation injury using the same criteria, have demonstrated the highest levels of inherent SH compounds. In addition, when mitotic cells were treated with cysteamine, they became very resistant to radiation. It has also been noted that cysteamine may directly protect cells against induced mutations. The protection is thought to result from scavenging of free radicals, either directly or via release of protein-bound GSH. An enzyme that liberates cysteamine from coenzyme A has been reported in avian liver and hog kidney. Recently, studies have appeared demonstrating a protective effect of cysteamine against the hepatotoxic agents acetaminophen, bromobenzene, and phalloidine.

Cystamine, in addition, to its role as a radioprotectant, has been found to alleviate tremors and prolong life in mice with the gene mutation for Huntington's disease (HD). The drug may work by increasing the activity of proteins that protect nerve cells, or neurons, from degeneration. Cystamine appears to inactivate an enzyme called transglutaminase and thus results in a reduction of huntingtin protein (Nature Medicine 8, 143-149, 2002). In addition, cystamine was found to increase the levels of certain neuroprotective proteins. However, due to the current methods and formulation of delivery of cystamine, degradation and poor uptake require excessive dosing.

At present, cysteamine is FDA approved only for the treatment of cystinosis. Patients with cystinosis are normally required to take cysteamine every 6 hours or use an enteric form of cysteamine (PROCYSBI®) every 12 hours. Subjects with cystinosis are required to ingest oral cysteamine (CYSTAGON®) every 6 hours day and night or use an enteric form of cysteamine (PROCYSBI®) every 12 hours. When taken regularly, cysteamine can deplete intracellular cystine by up to 90% (as measured in circulating white blood cells), and reduces the rate of progression to kidney failure/transplantation and also to obviate the need for thyroid replacement therapy. Because of the difficulty in taking CYSTAGON®, reducing the required dosing improves the adherence to therapeutic regimen. International Publication No. WO 2007/089670 demonstrates that delivery of cysteamine to the small intestine reduces gastric distress and ulceration, increases Cₘₐₓ and increases AUC. Delivery of cysteamine into the small intestine is useful due to improved absorption rates from the small intestine, and/or less cysteamine undergoing hepatic first pass elimination when absorbed through the small intestine. A decrease in leukocyte cystine was observed within an hour of treatment.

A pilot trial by Dohil et al. in 11 children with biopsy-confirmed non-alcoholic fatty liver disease (NAFLD) received enteric-coated (EC) cysteamine bitartrate orally for 24 weeks. This therapy resulted in statistically significant reductions in mean serum levels of alanine aminotransferase (ALT), aspartate aminotransferase (AST), total adiponectin, leptin, and cytokeratin-18 fragments, but without a concomitant reduction in body mass index. Seven out of 11 subjects reached the primary endpoints (of at least 50% reduction in ALT). The reduction in mean ALT and AST levels persisted 16 weeks after treatment ended.

In a particular embodiment, the disclosure provides for a compound having the structure of Formula I:

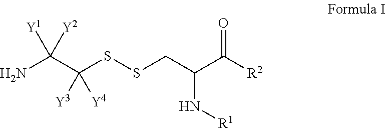

or a pharmaceutically acceptable salt or solvate thereof, wherein:
R¹ is selected from H or an acetyl group;
R² is selected from

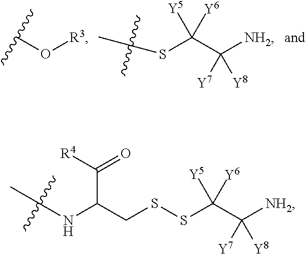

R³ is selected from an optionally substituted (C₁-C₆)alkyl, an optionally substituted cycloalkyl, an optionally substituted benzyl, or an optionally substituted aryl;

R⁴ is selected from

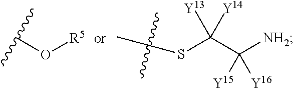

and
R⁵ is selected from an optionally substituted (C₁-C₆)alkyl, an optionally substituted cycloalkyl, an optionally substituted benzyl, or an optionally substituted aryl; and Y¹-Y¹⁶ are each independently selected from H or D.

In another embodiment, the disclosure provides for a compound having the structure of Formula II

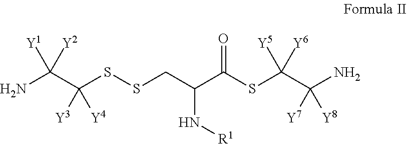

or a pharmaceutically acceptable salt or solvate thereof, wherein:
Y¹-Y⁸ are each independently selected from H or D; and
R¹ is selected from H or an acetyl group.

In another embodiment, the disclosure provides for a compound having the structure of Formula II(a):

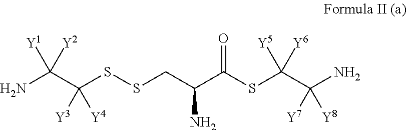

or a pharmaceutically acceptable salt or solvate thereof, wherein:
Y¹-Y⁸ are each independently selected from H or D.

In another embodiment, the disclosure provides for a compound having the structure selected from:

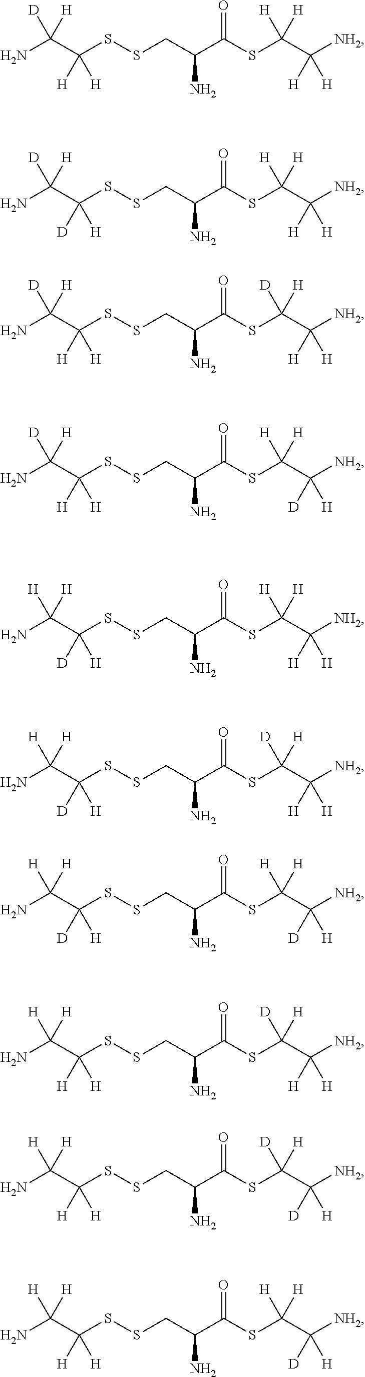

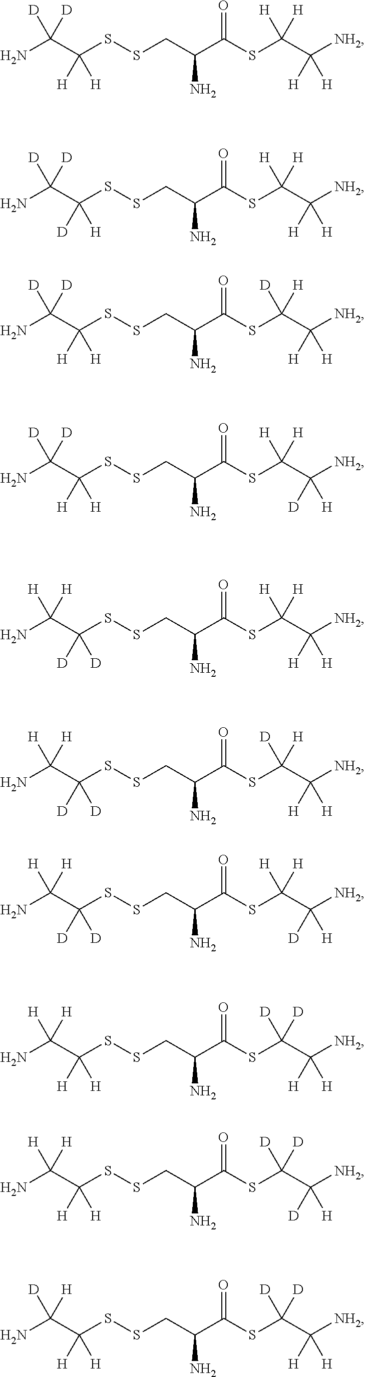

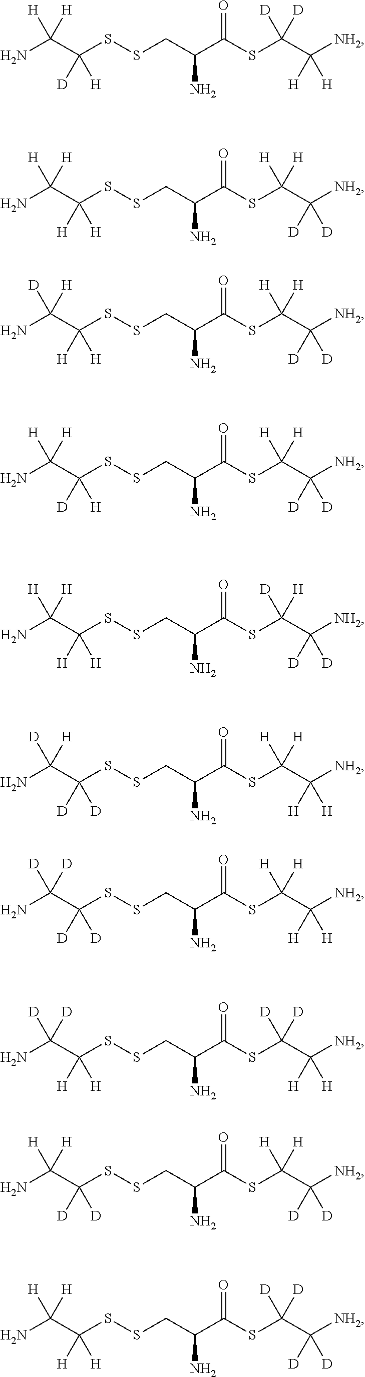

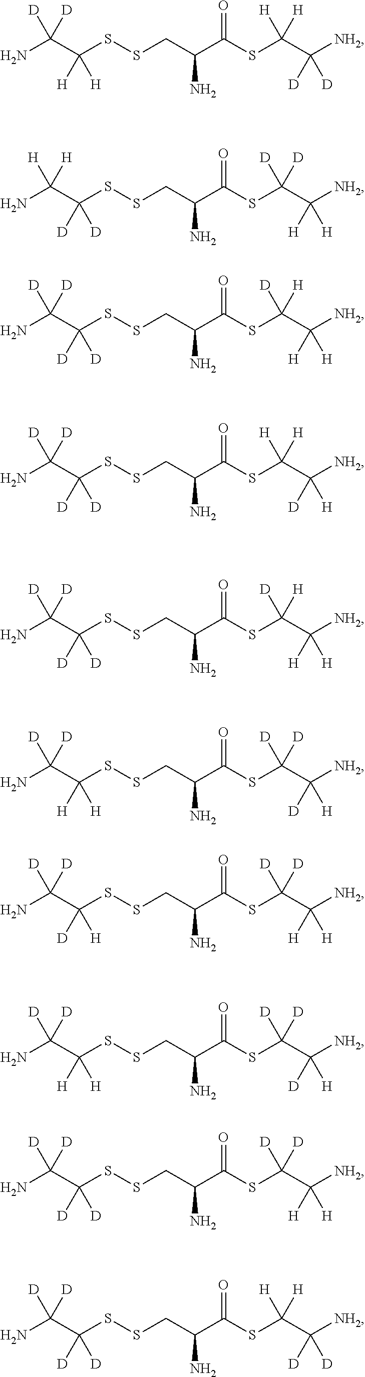

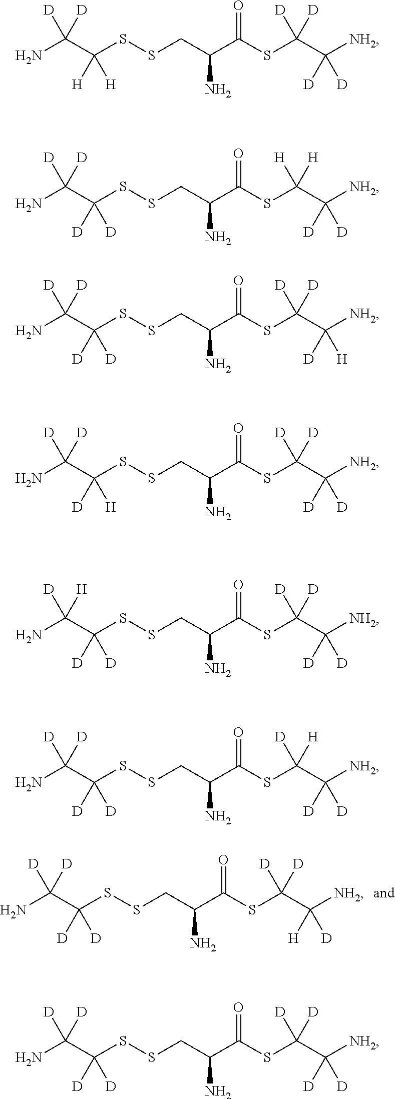

or a pharmaceutically acceptable salt or solvate of any one of the foregoing.

In a further embodiment, the pharmaceutically acceptable salt of Formula I has the structure of Formula II(b):

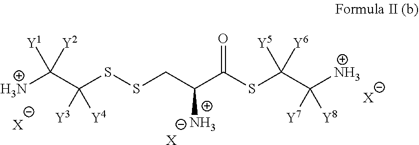

wherein,
Y¹-Y⁸ are each independently selected from H or D; and
X is a pharmaceutically acceptable counter ion.

In another embodiment, the disclosure provides for a compound having the structure of Formula II(c):

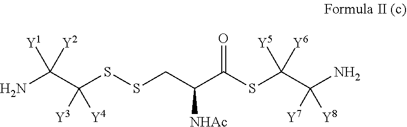

or a pharmaceutically acceptable salt or solvate thereof, wherein:
Y¹-Y⁸ are each independently selected from H or D.

In another embodiment, the disclosure provides for a compound having the structure selected from:

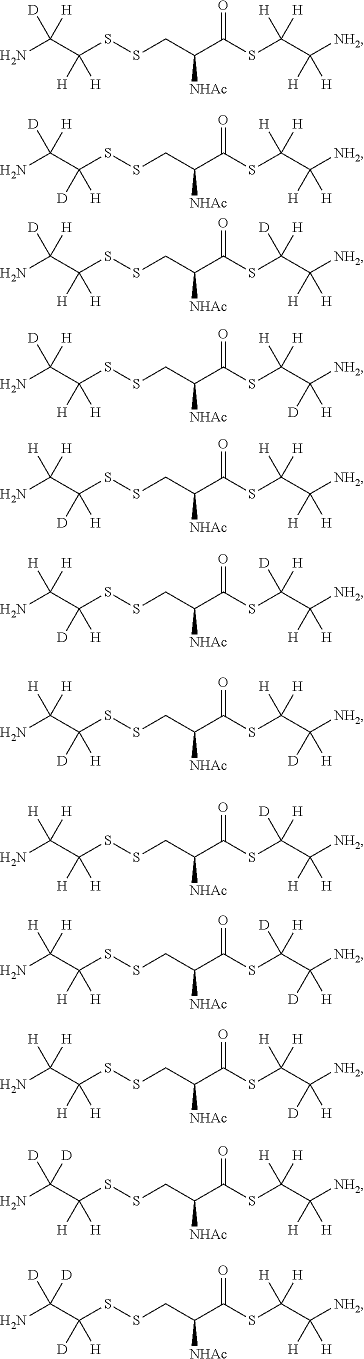

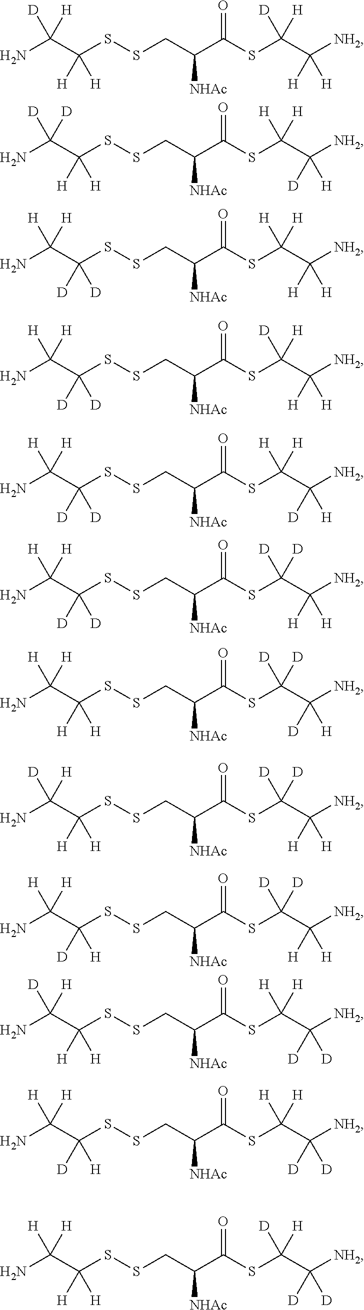

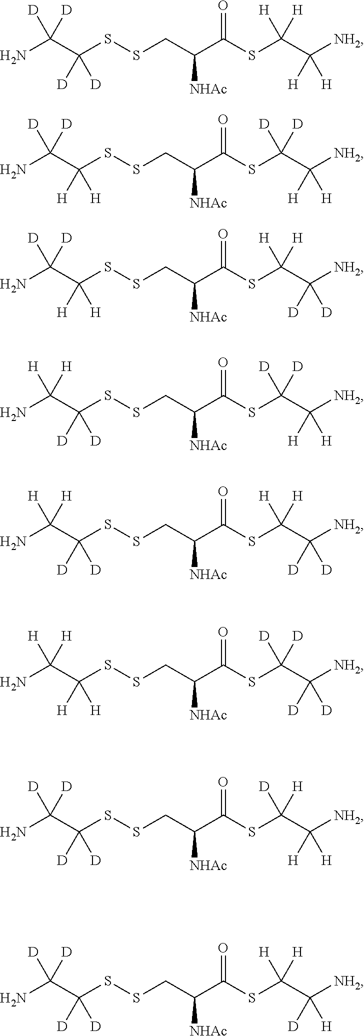

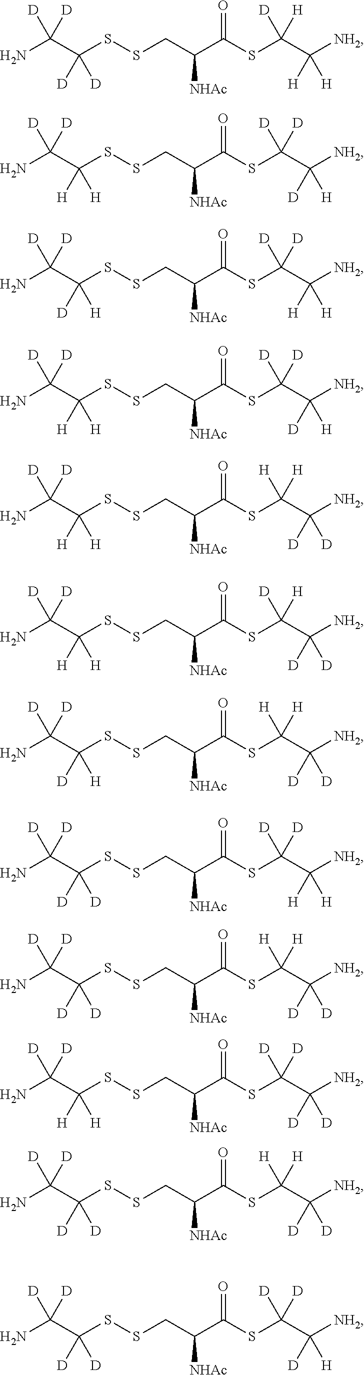

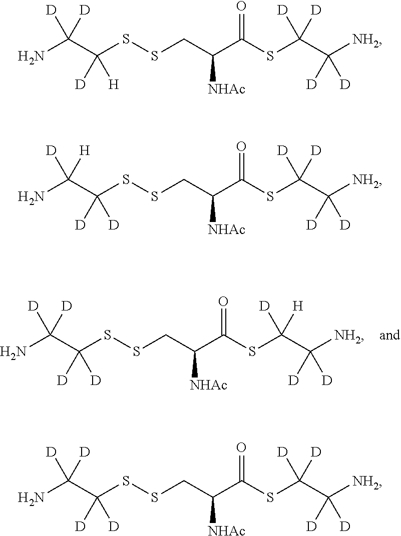 [SMILES: [2H]C(SSC[C@H](NC(C)=O)C(=O)SC([2H])([2H])C([2H])([2H])N)C([2H])([2H])N ; [2H]C(N)C([2H])([2H])SSC[C@H](NC(C)=O)C(=O)SC([2H])([2H])C([2H])([2H])N ; [2H]C(SC(=O)[C@H](CSSC([2H])([2H])C([2H])([2H])N)NC(C)=O)C([2H])([2H])N ; "and" ; [2H]C([2H])(N)C([2H])([2H])SSC[C@H](NC(C)=O)C(=O)SC([2H])([2H])C([2H])([2H])N]

or a pharmaceutically acceptable salt or prodrug thereof.

In a further embodiment, the pharmaceutically acceptable salt of Formula I has the structure of Formula II(d):

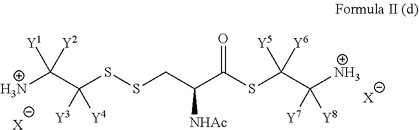

wherein,
Y¹-Y⁸ are each independently selected from H or D; and
X is a pharmaceutically acceptable counter ion.

In another embodiment, the disclosure provides for a compound having the structure of Formula III:

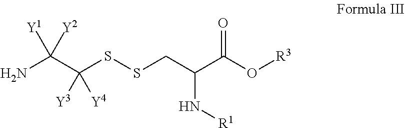

or a pharmaceutically acceptable salt or solvate thereof, wherein:
Y¹-Y⁴ are each independently selected from H or D;
R¹ is selected from H or an acetyl group;
R³ is a (C₁-C₆)alkyl.

In another embodiment, the disclosure provides for a compound having the structure of Formula III(a):

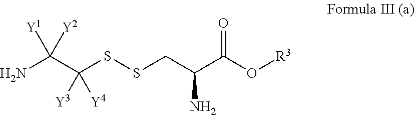

or a pharmaceutically acceptable salt or solvate thereof, wherein:
Y¹-Y⁴ are each independently selected from H or D; and
R³ is selected from a (C₁-C₆)alkyl.

In another embodiment, the disclosure provides for a compound having the structure selected from:

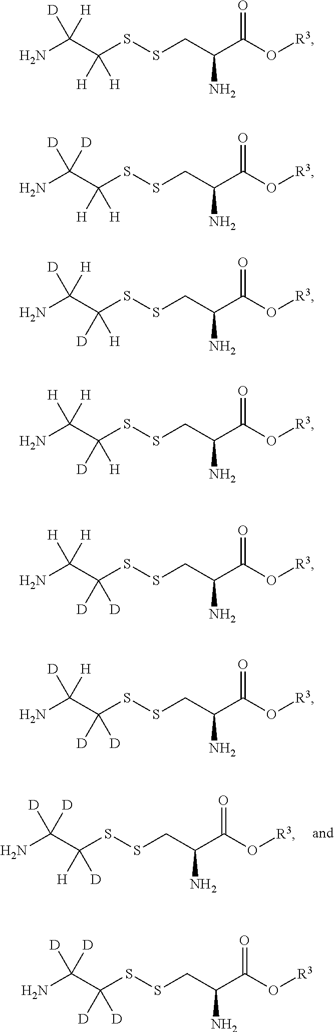

or a pharmaceutically acceptable salt or solvate of any one of the foregoing, wherein R³ is a (C₁-C₆)alkyl.

In a further embodiment, the pharmaceutically acceptable salt of Formula I has the structure of Formula III(b):

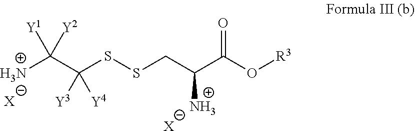

wherein,
Y¹-Y⁴ are each independently selected from H or D;
R³ is a (C₁-C₆)alkyl; and
X is a pharmaceutically acceptable counter ion.

In another embodiment, the disclosure provides for a compound having the structure of Formula III(c):

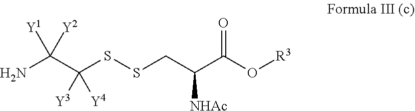

or a pharmaceutically acceptable salt or solvate thereof, wherein:
Y¹-Y⁴ are each independently selected from H or D; and
R³ is a (C₁-C₆)alkyl.

In another embodiment, the disclosure provides for a compound having the structure selected from:

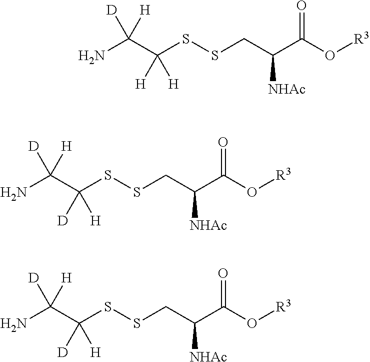

or a pharmaceutically acceptable salt or prodrug thereof, wherein
R³ is a (C₁-C₆)alkyl.

In a further embodiment, the pharmaceutically acceptable salt of Formula I has the structure of Formula III(d):

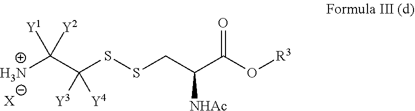

wherein,
Y¹-Y⁴ are each independently selected from H or D;
R³ is a (C₁-C₆)alkyl; and
X is a pharmaceutically acceptable counter ion.

In another embodiment, the disclosure provides for a compound having the structure of Formula IV:

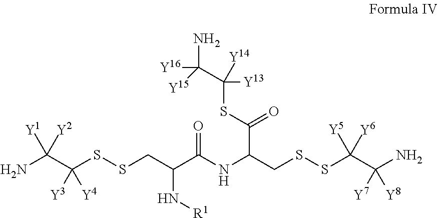

or a pharmaceutically acceptable salt or solvate thereof, wherein:
Y¹-Y⁴ and Y¹-Y¹⁶ are each independently selected from H or D; and
R¹ is selected from H or an acetyl group.

In another embodiment, the disclosure provides for a compound having the structure of Formula V:

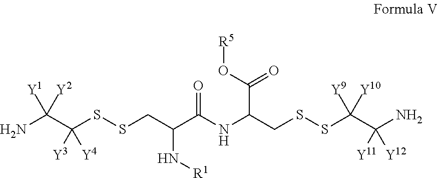

or a pharmaceutically acceptable salt or solvate thereof, wherein:
Y¹-Y⁴ and Y⁹-Y¹⁶ are each independently selected from H or D;
R¹ is selected from H or an acetyl group; and
R⁵ is a (C₁-C₆)alkyl.

In another embodiment, the disclosure provides for a compound having the structure of Formula VI:

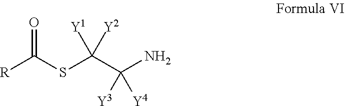

or a pharmaceutically acceptable salt or solvate thereof, wherein: Y¹-Y⁴ are each independently selected from H or D; and R is linear or branched aliphatic group (saturated or unsaturated) or aromatic (substituted or non-substituted) having from 1 to 20 carbon atoms. In another embodiment, at least one of Y¹-Y⁴ independently has deuterium enrichment of no less than about 10%. In yet another embodiment, at least one of Y¹-Y⁴ independently has deuterium enrichment of no less than about 50%. In a further embodiment, at least one of Y¹-Y⁴ independently has deuterium enrichment of no less than about 90%. In yet a further embodiment, at least one of Y¹-Y⁴ independently has deuterium enrichment of no less than about 98%.

In another embodiment, the disclosure provides for a compound having the structure of Formula VI(a):

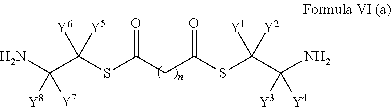

or a pharmaceutically acceptable salt or solvate thereof, wherein: Y¹-Y⁸ are each independently selected from H or D; and n is 2-6 (e.g., 2, 3, 4, 5, or 6). In another embodiment, at least one of Y¹-Y⁸ independently has deuterium enrichment of no less than about 10%. In yet another embodiment, at least one of Y¹-Y⁸ independently has deuterium enrichment of no less than about 50%. In a further embodiment, at least one of Y¹-Y⁸ independently has deuterium enrichment of no less than about 90%. In yet a further embodiment, at least one of Y¹-Y⁸ independently has deuterium enrichment of no less than about 98%.

In another embodiment, the disclosure provides for a compound having the structure selected from the group consisting of:

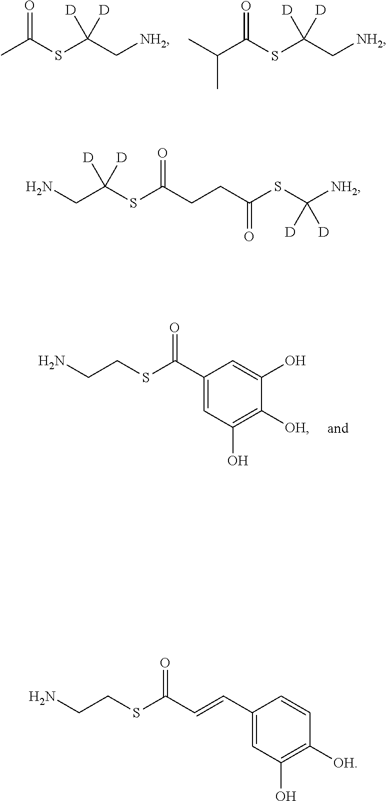

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) JU-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation,

$$k = {Ae}^{\frac{- {E}_{a}}{RT}} .$$

The Arrhenius equation states that, at a given temperature, the rate of a chemical reaction depends exponentially on the activation energy (Eₐ).

The transition state in a reaction is a short-lived state along the reaction pathway during which the original bonds have stretched to their limit. By definition, the activation energy Eₐ for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (H), a C-D bond is stronger than the corresponding C—H bond. If a C—H bond is broken during a rate-determining step in a chemical reaction (i.e., the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects.

Deuterium (D) is a stable and non-radioactive isotope of hydrogen which has approximately twice the mass of protium (H), the most common isotope of hydrogen. Deuterium oxide (DO) or deuterium dioxide (D₂O) or “heavy water” looks and tastes like H₂O, but has different physical properties. When pure D₂O is given to rodents, it is readily absorbed. The quantity of deuterium required to induce toxicity is extremely high. When about 0-15% of the body water has been replaced by heavy water, animals are healthy but are unable to gain weight as fast as the control (untreated) group. When about 15-20% of the body water has been replaced with heavy water the animals become excitable. When about 20-25% of the body water has been replaced with heavy water, the animals become so excitable that they go into frequent convulsions when stimulated. Skin lesions, ulcers on the paws and muzzles, and necrosis of the tails appear. The animals also become very aggressive. When about 30% of the body water has been replaced with heavy water, the animals refuse to eat and become comatose. Their body weight drops sharply and their metabolic rates drop far below normal, with death occurring at about 30 to about 35% replacement with heavy water. The effects are reversible unless more than thirty percent of the previous body weight has been lost due to heavy water. Studies have also shown that the use of heavy water can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetylchloride. However, this method may not be applicable to all drug classes. For example, deuterium incorporation can lead to Metabolic Switching. Metabolic Switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

The compounds described herein which comprise deuterium atoms are expected to prevent or retard metabolism of the compounds. Other sites on the molecule may also undergo transformations leading to metabolites with as-yet unknown pharmacology/toxicology. Limiting the production of such metabolites has the potential to decrease the danger of the administration of such drugs and may even allow increased dosage and concomitant increased efficacy. All of these transformations, among other potential transformations, can occur through polymorphically-expressed enzymes, leading to interpatient variability. Further, it is quite typical for disorders ameliorated by the compositions and methods of the disclosure, such as NAFLD, NASH or cystinosis, to produce symptoms that are best medicated around the clock for extended periods of time.

For all of the foregoing reasons, a medicine with a longer half-life can provide greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. Compounds of the disclosure which comprise deuterium atoms have the potential to slow the metabolism and/or selectively shunt the metabolism of the compounds to more favorable enzymatic pathways. For example, it is expected that the compounds comprising deuterium atoms presented herein could potentially prevent or reduce the production of odiferous cysteamine metabolites that can lead to patient noncompliance.

The disclosure provides bioprotective compounds and pharmaceutical compositions have been discovered, together with methods of synthesizing and using the compounds, including methods for the treatment of liver diseases and disorders in a patient by administering a compound of the disclosure.

The disclosure is not limited with respect to a specific salt form of Formula I (e.g., the disclosure is not limited to any specific pharmaceutically acceptable salt). Further, the pharmaceutical compositions of the disclosure can contain a compound of the disclosure individually, or combination of compounds of the disclosure, where one or both compounds are deuterated. The active agents in the composition, i.e., the compounds of the disclosure, may be administered in the form of a pharmacologically acceptable salt or solvate thereof. Salts and solvates of the compounds may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, basic addition salts are prepared from the neutral drug using conventional means, involving reaction of one or more of the active agent's free hydroxyl groups with a suitable base. Generally, the neutral form of the drug is dissolved in a polar organic solvent such as methanol or ethanol and the base is added thereto. The resulting salt either precipitates or may be brought out of solution by addition of a less polar solvent. Suitable bases for forming basic addition salts include, but are not limited to, inorganic bases such as sodium hydroxide, potassium hydroxide, ammonium hydroxide, calcium hydroxide, trimethylamine, or the like.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% DO or about 0.00001% DHO, assuming that all of the C-D bonds in the compound as disclosed herein are metabolized and released as DO or DHO. In certain embodiments, the levels of DO shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of DO or DHO upon drug metabolism.

In a further embodiment, the compounds of the disclosure exhibit a reduced rate of metabolism by at least one polymorphically-expressed cytochrome P450 isoform in a subject per dosage unit thereof in comparison to non-isotopically enriched cysteamine and cystamine. Examples of polymorphically-expressed cytochrome P450 isoforms include, but are not limited to, CYP2C8, CYP2C9, CYP2C19, and CYP2D6. In another embodiment, the compounds of the disclosure exhibit a reduced rate of metabolism by at least one cytochrome P450 isoform or monoamine oxidase isoform in a subject per dosage unit thereof in comparison to non-isotopically enriched cysteamine and cystamine. Examples of cytochrome P450 isoforms and monoamine oxidase isoforms, include but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAOA, and MAOB.

In certain embodiments, the compounds of the disclosure exhibit an improvement in a diagnostic hepatobiliary function end point, as compared to the corresponding non-isotopically enriched cysteamine and cystamine. Examples of diagnostic hepatobiliary function endpoints include, but are not limited to, alanine aminotransferase (ALT), serum glutamic pyruvic transaminase (“SGPT), aspartate aminotransferase (“AST”, “SGOT”), ALT/AST ratios, serum aldolase, alkaline phosphatase (ALP), ammonia levels, bilirubin, gamma glutamyltranspeptidase (“GGTP”, “γ-GTP”, “GGT”), leucine aminopeptidase (“LAP”), liver biopsy, liver ultrasonography, liver nuclear scan, 5′-nucleotidase, and blood protein.

As shown in the results presented herein, the compounds of the disclosure exhibited nearly identical cystine depletion kinetics as cystamine. Further, cystine reaccumulated with a compound of the disclosure following washout at a rate similar to previously described with cystamine. Suggesting that both drugs were acting in a similar way in depleting intracellular cystine.

Additional experiments presented herein, the intracellular reduction of a compound disclosed herein to cysteamine was quite rapid and similar to cystamine. Additionally, the intracellular levels of cystamine are higher following cystamine administration in comparison to a compound disclose herein, as the compound is not required to be reduced before detection. The release of cysteine was higher with the compound of the disclosure than with cystamine. Interestingly this does not impact the level of cystine depletion during continuous drug exposure or the degree of cystine accumulation following drug washout.

Based upon the studies, it is clear that the compounds disclosed herein make more cysteine available intracellularly in comparison to cysteamine or cystamine. Glutathione was induced and at the same levels when cystamine or a compound of the disclosure were administered.

In another embodiment, processes for preparing a compound as disclosed herein or other pharmaceutically acceptable derivative thereof such as a salt, solvate, or prodrug, as an antioxidant, and a treatment for cystinosis and fatty liver disorders, such as NAFLD and NASH.

While it may be possible for the compounds of the disclosure to be administered as the raw chemical, it is also possible to present them as a pharmaceutical composition. Accordingly, provided herein are pharmaceutical compositions which comprise one or more of certain deuterated compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington. The Science and Practice of Pharmacy, supra; Modified-Release Drug Delivery Technology, Rathbone et al., Eds. Drugs and the Pharmaceutical Science, Marcel Dekker, Inc.: New York, N.Y., 2002; Vol. 126). For example, in one embodiment, a deuterated cysteamine and/or cystamine can be enterically coated (e.g., enterically coated beads or capsules). As mentioned above and elsewhere herein non-deuterated enteric formulations of cysteamine bitartrate have been shown to provide improved drug compliance, reduce frequency of administration and prolonged reduction of cystine levels in cystinosis patients. Because the deuterated forms of the compounds of the disclosure also include a longer half-life, an enterically coated formulation comprising a deuterated compound of formula I and/or II can have improved administration and longer biological activity. In some embodiments, an enterically coated formulation of compound of formula I and/or II can be administered at lower doses than a non-deuterated enteric formulation and/or may be administered less frequently.

The compositions include those suitable for oral, parenteral (including Subcutaneous, intradermal, intramuscular, intravenous, intraarticular, and intramedullary), intraperitoneal, transmucosal, transdermal, rectal and topical (including dermal, buccal, Sublingual and intraocular) administration. The most suitable route for administration depends on a variety of factors, including interpatient variation or disorder type, and therefore the disclosure is not limited to just one form of administration. The compositions may conveniently be presented in unit dosage form and may be prepared by any of the methods well known in the art of pharmacy. Typically, these methods include the step of bringing into association a compound of the disclosure or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules (including enterically coated granules); as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Moulded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer Solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Typical unit dosage formulations are those containing an effective dose, as herein below recited, or an appropriate fraction thereof, of the active ingredient.

Tablets or other forms of presentation provided in discrete units may conveniently contain an amount of one or more compounds which is effective at such dosage or as a multiple of the same, for instance, units containing 1 mg to 1000 mg of the compounds disclosed herein, usually around 100 mg to 500 mg of the compound.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

The disclosure contemplates that typical delivery will be by oral routes. Formulations for such delivery include enterically coated formulations as well as non-enterically formulated formulation comprising at least one deuterated form of cystamine and/or cysteamine. As presented below, the deuterated forms comprise pharmacokinetic and pharmacodynamic changes related to non-deuterated forms. Moreover, prior formulations comprising enterically coated cysteamine and/or cystamine have also showed improved pharmacokinetic and pharmacodynamic data relative to non-enterically formulated formulations. Accordingly, the combination of enterically coated and deuterated forms of cystamine and/or cysteamine are expected to further modulate the pharmacokinetics and pharmacodynamics of cysteamine and/or cysteamine delivery including, for example, both the delayed and extended release of the active ingredient as reflected by modulation of the AUC and Cₘₐₓ and/or Tₘₐₓ.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday’). Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

This disclosure identifies patient populations that can benefit from the compounds disclosed herein, particularly juvenile patients. The disclosure provides composition of the compounds disclosed herein that can be used in the treatment of various diseases including cystinosis, Huntington's disease, and NAFLD (including NASH).

Cystinosis is a rare disease that is typically diagnosed prior to age 2. Cystinosis is a genetic metabolic disease that causes an amino acid, cystine, to accumulate in various organs of the body. Cystine crystals accumulate in the kidneys, eyes, liver, muscles, pancreas, brain, and white blood cells. Without specific treatment, children with cystinosis develop end stage kidney failure at approximately age nine. Cystinosis also causes complications in other organs of the body. The complications include muscle wasting, difficulty swallowing, diabetes, and hypothyroidism. It is estimated that at least 2,000 individuals worldwide have cystinosis, though exact numbers are difficult to obtain because the disease is often undiagnosed and/or misdiagnosed. There are three forms of Cystinosis. Infantile Nephropathic Cystinosis is the most severe form of the disease. Children with Cystinosis appear normal at birth, but by 10 months of age, they are clearly shorter than others their age. They urinate frequently, have excessive thirst, and often seem fussy. At 12 months, they haven't walked and bear weight only gingerly. One of the major complications of Cystinosis is renal tubular Fanconi Syndrome, or a failure of the kidneys to reabsorb nutrients and minerals. The minerals are lost in the urine. The urinary losses must be replaced. Generally, they are picky eaters, crave salt, and grow very slowly. If left untreated, this form of the disease may lead to kidney failure by 10 years of age. In people with Intermediate Cystinosis or Juvenile (adolescent) Cystinosis, kidney and eye symptoms typically become apparent during the teenage years or early adulthood. In Benign or Adult Cystinosis, cystine accumulates primarily in the cornea of the eyes. Cystinosis is treated symptomatically. Renal tubular dysfunction requires a high intake of fluids and electrolytes to prevent excessive loss of water from the body (dehydration). Sodium bicarbonate, sodium citrate, and potassium citrate may be administered to maintain the normal electrolyte balance. Phosphates and vitamin D are also required to correct the impaired uptake of phosphate into the kidneys and to prevent rickets. Carnitine may help to replace muscular carnitine deficiency.

Cysteamine (Cystagon®) has been approved by the Food and Drug Administration (FDA) for standard treatment of Cystinosis. Cysteamine is a cystine-depleting agent that lowers cystine levels within the cells. Cysteamine has proven effective in delaying or preventing renal failure. Cysteamine also improves growth of children with Cystinosis. In view of the harmful effects of chronic cystine accumulation, and the indications of the effectiveness of Cysteamine therapy in various tissues and organ systems, oral Cysteamine should be used by post-transplant Cystinosis patients. Procysbi® (cysteamine bitartrate delayed release capsules) was approved by the FDA in May 2013. Cystaran (cysteamine ophthalmic solution) 0.44% is an ophthalmic solution approved by the FDA for the treatment of corneal cystine crystal accumulation in patients with cystinosis.

In one embodiment, a subject having cystinosis is administered a compound of the disclosure or pharmaceutically acceptable salt thereof in an amount to obtain about 10-200 μmol (e.g., 10, 20, 30, 40, 50, 60, 70, 80, 90, 100, 110, 120, 130, 140, 150, 160, 170, 180, 190, 200, or any value there between) of the compound in the plasma. In one embodiment, the dose is up to about 10-95 mg/kg. In another embodiment, the dose is administered 2-4 times per day at about 100 mg to 1 gram per dose. In a further embodiment, the compound of the disclosure is administered in multiple doses that do not exceed 2.0 g/m²/day or 95 mg/kg/day. When the compound is well tolerated, the goal of therapy is to keep leukocyte cystine levels below 1 nmol/½ cystine/mg protein five to six hours following administration of the compound of the disclosure. Patients with poorer tolerability still receive significant benefit if white cell cystine levels are below 2 nmol/½ cystine/mg protein. The dose of the compound disclosed herein can be increased to a maximum of 2.0 grams/m²/day to achieve this level

Patients over age 12 and over 110 pounds should receive 2.0 grams/day given in four divided doses as a starting maintenance dose. This dose should be reached after 4 to 6 weeks of incremental dosage increases as stated above. The dose should be raised if the leukocyte cystine level remains >2 nmol/½ cystine/mg/protein.

Leukocyte cystine measurements, taken 5 to 6 hours after dose administration, are recommended for new patients after the maintenance dose is achieved. Patients being transferred from solutions comprising the compound to capsules should have their white cell cystine levels measured in 2 weeks, and thereafter every 3 months to assess optimal dosage as described above.

If the compound of the disclosure is poorly tolerated initially due to gastrointestinal tract symptoms or transient skin rashes, therapy should be temporarily stopped, then re-instituted at a lower dose and gradually increased to the proper dose.

The compositions and methods of the disclosure can also be used to treat NAFLD and NASH as well as liver fibrotic diseases. Non-alcoholic fatty liver disease (NAFLD) represents a spectrum of disease occurring in the absence of alcohol abuse. It is characterized by the presence of steatosis (fat in the liver) and may represent a hepatic manifestation of the metabolic syndrome (including obesity, diabetes and hypertriglyceridemia). NAFLD is linked to insulin resistance, it causes liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90). NASH is characterized by the histologic presence of steatosis, cytological ballooning, scattered inflammation and pericellular fibrosis (Contos et al., Adv Anat Pathol 2002; 9:37-51). Hepatic fibrosis resulting from NASH may progress to cirrhosis of the liver or liver failure, and in some instances may lead to hepatocellular carcinoma.

The degree of insulin resistance (and hyperinsulinemia) correlates with the severity of NAFLD, being more pronounced in patients with NASH than with simple fatty liver (Sanyal et al., Gastroenterology 2001; 120(5):1183-92). As a result, insulin-mediated suppression of lipolysis occurs and levels of circulating fatty acids increase. Two factors associated with NASH include insulin resistance and increased delivery of free fatty acids to the liver. Insulin blocks mitochondrial fatty acid oxidation. The increased generation of free fatty acids for hepatic re-esterification and oxidation results in accumulation of intrahepatic fat and increases the liver's vulnerability to secondary insults.

Glutathione (gammaglutamyl-cysteinyl-glycine; GSH) is a major endogenous antioxidant and its depletion is implicated in the development of hepatocellular injury (Wu et al., J Nutr 2004; 134(3):489-92). One such injury is acetaminophen poisoning, where reduced GSH levels become depleted in an attempt to conjugate and inactivate the hepatotoxic metabolite of the drug. After a toxic dose of acetaminophen, excess metabolite (N-acetyl-benzoquinoneimine) covalently binds to hepatic proteins and enzymes resulting in liver damage (Wu et al., J Nutr 2004; 134(3):489-92; Prescott et al., Annu Rev Pharmacol Toxicol 1983; 23:87-101). Increased glutathione levels appears therefore to have some protective effects through the reduction of ROS. Glutathione itself is does not enter easily into cells, even when given in large amounts. However, glutathione precursors do enter into cells and some GSH precursors such as N-acetylcysteine have been shown to be effective in the treatment of conditions such as acetaminophen toxicity by slowing or preventing GSH depletion (Prescott et al., Annu Rev Pharmacol Toxicol 1983; 23:87-101). Examples of GSH precursors include cysteine, N-acetylcysteine, methionine and other sulphur-containing compounds such as cysteamine (Prescott et al., J Int Med Res 1976; 4(4 Suppl):112-7).

Cysteine is a major limiting factor for GSH synthesis and that factors (e.g., insulin and growth factors) that stimulate cysteine uptake by cells generally result in increased intracellular GSH levels (Lyons et al., Proc Natl Acad Sci USA 2000; 97(10):5071-6; Lu S C. Curr Top Cell Regul 2000; 36:95-11).

N-acetylcysteine has been administered to patients with NASH. In reports from Turkey, obese individuals with NASH treated with N-acetylcysteine for 4-12 weeks exhibited an improvement in aminotransferase levels and gamma-GT even though there was no reported change in subject body mass index (Pamuk et al., J Gastroenterol Hepatol 2003; 18(10):1220-1).

Studies in mice and humans showed cysteamine to be effective in preventing acetaminophen-induced hepatocellular injury (Prescott et al., Lancet 1972; 2(7778):652; Prescott et al., Br Med J 1978; 1(6116):856-7; Mitchell et al., Clin Pharmacol Ther 1974; 16(4):676-84). Cystamine and cysteine have been reported to reduce liver cell necrosis induced by several hepatotoxins. (Toxicol Appl Pharmacol. 1979 April; 48 (2):221-8). Cystamine has been shown to ameliorate liver fibrosis induced by carbon tetrachloride via inhibition of tissue transglutaminase (Qiu et al., World J Gastroenterol. 13:4328-32, 2007).

The prevalence of NAFLD in children is unknown because of the requirement of histologic analysis of liver in order to confirm the diagnosis (Schwimmer et al., Pediatrics 2006; 118 (4):1388-93). However, estimates of prevalence can be inferred from pediatric obesity data using hepatic ultra-sonongraphy and elevated serum transaminase levels and the knowledge that 85% of children with NAFLD are obese. Data from the National Health and Nutrition Examination Survey has revealed a threefold rise in the prevalence of childhood and adolescent obesity over the past 35 years; data from 2000 suggests that 14-16% children between 6-19 yrs age are obese with a BMI >95% (Fishbein et al., J Pediatr Gastroenterol Nutr 2003; 36(1):54-61), and also that fact that 85% of children with NAFLD are obese.

The exact mechanism by which NAFLD develops into NASH remains unclear. Because insulin resistance is associated with both NAFLD and NASH, it is postulated that other additional factors are also required for NASH to arise. This is referred to as the “two-hit” hypothesis (Day C P. Best Pract Res Clin Gastroenterol 2002; 16(5):663-78) and involves, firstly, an accumulation of fat within the liver and, secondly, the presence of large amounts of free radicals with increased oxidative stress. Macrovesicular steatosis represents hepatic accumulation of triglycerides, and this in turn is due to an imbalance between the delivery and utilization of free fatty acids to the liver. During periods of increased calorie intake, triglyceride will accumulate and act as a reserve energy source. When dietary calories are insufficient, stored triglycerides (in adipose) undergo lipolysis and fatty acids are released into the circulation and are taken up by the liver. Oxidation of fatty acids will yield energy for utilization. Treatment of NASH currently revolves around the reduction of the two main pathogenetic factors, namely, fat accumulation within the liver and excessive accumulation of free radicals causing oxidative stress. Fat accumulation is diminished by reducing fat intake as well as increasing caloric expenditure. One therapeutic approach is sustained and steady weight loss. Although not definitively proven, a >10% loss in body weight has been shown in some cases to reduce hepatic fat accumulation, normalize liver transaminases and improve hepatic inflammation and fibrosis (Ueno et al., J Hepatol 1997, 27(1):103-7; Vajro et al., J Pediatr 1994; 125(2):239-41; Franzese et al., Dig Dis Sci 1997, 42(7):1428-32).

Reduction of oxidative stress through treatment with antioxidants has also been shown to be effective in some studies. For example, obese children who had steatosis were treated with vitamin E (400-1000 IU/day) for 4-10 months (Lavine, J Pediatr 2000, 136(6):734-8). Despite any significant change in BMI, the mean ALT levels decreased from 175±106 IU/L to 40±26 IU/L (P<0.01) and mean AST levels decreased from 104±61 IU/L to 33±11 IU/L (P<0.002). Hepatic transaminases increased in those patients who elected to discontinue vitamin E therapy. An adult study using vitamin E for one year demonstrated similar reduction of hepatic transaminases as well as the fibrosis marker TGFβ levels (Hasegawa et al., Aliment Pharmacol Ther 2001, 15(10):1667-72).

Steatosis also may develop into steatohepatitis through oxidative stress due to reactive oxygen species (ROS) and decreased anti-oxidant defense (Sanyal et al., Gastroenterology 2001, 120(5):1183-92). ROS can be generated in the liver through several pathways including mitochondria, peroxisomes, cytochrome P450, NADPH oxidase and lipooxygenase (Sanyal et al., Nat Clin Pract Gastroenterol Hepatol, 2005; 2(1):46-53). Insulin resistance and hyperinsulinism has been shown to increase hepatic oxidative stress and lipid peroxidation through increased hepatic CYP2EI activity (Robertson et al., Am J Physiol Gastrointest Liver Physiol, 2001 281(5):G1135-9; Leclercq et al., J Clin Invest 2000, 105(8):1067-75).

Currently, much of what is understood of the pathogenesis of NAFLD has arisen from animal studies. A number of mouse models which exhibit steatosis/steatohepatitis exist and include genetically altered leptin-deficient (ob/ob) or leptin resistant (db/db) and the dietary methionine/choline deficient (MCD) model. Studies comparing male and female rats of varying strains (Wistar, Sprague-Dawley, Long-Evans) with a mouse strain (C57BL/6) as models for NASH have been undertaken. These animals were fed for 4 weeks with an MCD diet; although ALT elevation and steatosis were more noticeable in the Wistar rat, the overall histologic changes in the liver of the mice were more constant with changes due to NASH. More recently the use of supra-nutritional diets in animals has resulted in a NAFLD model that physiologically more resembles the human phenotype. The medical conditions most commonly associated with NAFLD are obesity, Type II diabetes and dyslipidemia. These conditions can be induced by feeding mice and rats with high fat or sucrose diets. Rats fed with a >70% fat-rich diet for 3 weeks developed pan-lobular steatosis, patchy inflammation, enhanced oxidative stress, and increased plasma insulin concentrations suggesting insulin resistance. NASH mice have been induced through intragastric overfeeding. Mice were fed up to 85% in excess of their standard intake for 9 weeks. The mice became obese with 71% increase in final body weight; they demonstrated increase white adipose tissue, hyperglycemia, hyperinsulinemia, hyperleptinemia, glucose intolerance and insulin resistance. Of these mice 46% developed increased ALT (121=/−27 vs 13+/−1 U/L) as well as histologic features suggestive of NASH. The livers of the overfed mice were about twice as large expected, beige in color with microscopic evidence of lipid droplets, cytoplasmic vacuoles and clusters of inflammation.

Mouse models of NASH can be used to study various therapies. Mouse models are created through specific diets (methionine choline deficient, MCD) or intragastric overfeeding. These mice develop serologic and histologic features of NASH. NASH mice are useful in screening and measuring the effects cysteamine on NASH related disease and disorders. For example, the effect of treatment can be measured by separating the NASH mice into a control group where animals will continue to receive MCD diet only and three other treatment groups where mice will receive MCD diet as well as anti-oxidant therapy. The three therapy groups for example, can receive cysteamine 50 mg/kg/day, 100 mg/kg/day and sAME.

As mentioned above, NASH is a disease subset falling under the umbrella of NAFLD and is characterized by various biomarkers and histological examination. NASH has been characterized as including two types: Type 1 and Type 2, having some distinct biomarker and histological characteristics, while certain others that overlap between the two types. These two types, Type 1 and Type 2 NASH are typically identified in juvenile patients.

Type 1 NASH is characterized by steatosis, lobular inflammation, ballooning degeneration and perisinusoidal fibrosis. Type 2 NASH is characterized by steatosis, portal inflammation, and portal fibrosis. Schwimmer et al. (Hepatology, 42(3):641-649, 2005; incorporated herein by reference) described various criteria and biomarkers used to differentiate NASH Type 1 from NASH Type 2. In particular, Schwimmer et al. discloses that subjects with NASH Type 1 had higher AST, ALT and triglyceride levels compared to patients with NASH Type 2. However, the strongest factor demonstrating a difference in the two types of NASH are best found upon histological examination. As stated above, Type 1 NASH demonstrates a prevalent lobular inflammation in the liver in contrast with a prevalent portal inflammation in Type 2 NASH. Thus, the disclosure contemplates that one of the key differentiating factors that can be used in the methods disclosed herein is identifying, by histological examination, the presence of Type 1 vs. Type 2 NASH.

The diagnosis of steatosis is typically made when lipid deposition is visible in more than 5% of hepatocytes. NASH is diagnosed when, in addition to hepatic steatosis, both inflammatory infiltrates as well as ballooning and liver cell injury are present. The NAFLD Activity Score (NAS) was developed to provide a numerical score for patients who most likely have NASH. Accordingly, the NAS is the sum of the separate scores for steatosis (0-3), hepatocellular ballooning (0-2) and lobular inflammation (0-3), with the majority of patients with NASH having a NAS score of ≥5 (Kleiner D E, Brunt E M, Van Natta M et al. Design and validation of a histological scoring system for nonalcoholic fatty liver disease. Hepatology 41(6), 1313-1321 (2005)).

In addition, various studies have shown that cytokeratin 18 is a useful indicator of inflammation in NASH, due to cytokeratin 18's release from hepatocytes undergoing apoptosis. Normal cytokeratin 18 levels are typically characterized as being less than 200 units per liter. In contrast, subjects with liver disease, including NALFD and NASH have a statistically significant elevation in cytokeratin 18 (e.g., above 200 U/L; 200-300 U/L). Moreover, cytokeratin 18 levels can be used as a marker to determine whether a treatment is being effective. For example, a reduction in cytokeratin 18 levels of greater than 10% (e.g., 20-30%, 30-40%, 40-50%, 50-60%, 60-70%, 70-80% or 90-100%) is indicative that the therapy is having a beneficial effect. Other markers include commonly used liver function tests including measuring one or more of, for example, serum alanine aminotransferase (ALT), alkaline phosphatase (ALP), aspartate aminotransferase (AST) and gamma-glutamyl transpeptidase (GGT).

The effectiveness of a method or composition of the disclosure can be assessed by measuring fatty acid content and metabolism in the liver. Dosage adjustment and therapy can be made by a medical specialist depending upon, for example, the severity of NAFL.

In patients with histologically proven NAFLD, serum hepatic aminotransferases, specifically alanine aminotransferase (ALT), levels are elevated from the upper limit of normal to 10 times this level (Schwimmer et al., J Pediatr 2003, 143(4):500-5; Rashid et al., J Pediatr Gastroenterol Nutr 2000, 30(1):48-53). The ratio of ALT/AST (aspartate aminotransferase) is >1 (range 1.5-1.7) which differs from alcoholic steatohepatitis where the ratio is generally <1. Other abnormal serologic tests that may be abnormally elevated in NASH include gamma-glutamyltransferase (gamma-GT) and fasting levels of plasma insulin, cholesterol and triglyceride.

ALT levels have been shown to be indicative of liver function. For example, normal ALT levels are about 7 to 55 units (e.g., 10-40 units) per liter has been shown to correlate with normal liver function. This value is somewhat varied in children and adolescents. Thus, in some instances ALT levels less than 25 units per liter are “normal” in children and adolescents. Increased levels of ALT have been shown to correlate with liver disease and disorders. For example, NAFLD and NASH subjects typically show ALT levels of between 60 to 150 (e.g., 60-145, 70-140, 80-135, 90-130, 105-125, 110-120, or any number between any two values thereof). In some embodiment, particularly with children and adolescents, ALT levels above 25 units per liter can be indicative of NASH or NAFLD. In determining if a subject has NAFLD or NASH or is susceptible to treatment using a compound of the disclosure, ALT may be measured alone, but preferably, the determination should be made in combination with one or more other markers of liver function or dysfunction. For example, a subject having ALT levels above about 80 is indicative of liver disease or dysfunction.

AST levels have been shown to be indicative of liver function. For example, AST levels between about 8 to 48 units (e.g., 10-40 units) per liter has been shown to correlate with normal liver function. Increased levels of AST have been shown to correlate with liver disease and disorders. For example, NAFLD and NASH subjects typically show AST levels of between 40 to 100 (e.g., 45-95, 55-90, 65-85, 70-80, or any number between any two values thereof). In determining if a subject has NAFLD or NASH or is susceptible to treatment using a deuterated cysteamine or cystamine composition, AST may be measured alone, but preferably the determination should be made in combination with one or more other markers of liver function or dysfunction. For example, a subject having AST levels above about 50 is indicative of liver disease or dysfunction.

ALP levels have been shown to be indicative of liver function. For example, ALP levels between about 45 to 115 units (e.g., 50-110 units) per liter has been shown to correlate with normal liver function. Increased levels of ALP have been shown to correlate with liver disease and disorders. For example, NAFLD and NASH subjects typically show ALP levels of between 150 to 250 (e.g., 155-245, 160-240, 165-235, 170-230, 175-225, 180-220, 185-215, 190-210, 195-200, or any number between any two values thereof). In determining if a subject has NAFLD or NASH or is susceptible to treatment using a deuterated cysteamine or cystamine composition, ALP may be measured alone, but preferably the determination should be made in combination with one or more other markers of liver function or dysfunction. For example, a subject having ALP levels above about 150 is indicative of liver disease or dysfunction.

GGT levels have been shown to be indicative of liver function. For example, GGT levels between about 9 to 48 units (e.g., 10-40 units) per liter has been shown to correlate with normal liver function. Increased levels of GGT have been shown to correlate with liver disease and disorders. For example, NAFLD and NASH subjects typically show GGT levels of between 50 to 100 (e.g., 55-95, 60-90, 65-85, 70-80, or any number between any two values thereof). In determining if a subject has NAFLD or NASH or is susceptible to treatment using a deuterated cysteamine or cystamine composition, GGT may be measured alone, but preferably the determination should be made in combination with one or more other markers of liver function or dysfunction. For example, a subject having GGT levels above about 50 is indicative of liver disease or dysfunction.

Triglycerides levels have been shown to be indicative of liver function. For example, triglyceride levels less than about 150 mg/dL (e.g., 100-150 mg/dL) has been shown to correlate with normal liver function. Increased levels of triglycerides have been shown to correlate with liver disease and disorders. For example, NAFLD and NASH subjects typically show triglyceride levels of between 150 to 200 (e.g., 155-195, 160-190, 165-185, 170-180, or any number between any two values thereof). In determining if a subject has NAFLD or NASH or is susceptible to treatment using a deuterated cysteamine or cystamine composition, triglycerides may be measured alone, but preferably the determination should be made in combination with one or more other markers of liver function or dysfunction. For example, a subject having triglyceride levels above about 150 mg/dl is indicative of liver disease or dysfunction.

High triglyceride levels are known to be a leading cause of various forms of inflammation. Triglycerides are the form in which fat moves through the bloodstream. Triglycerides can be metabolized by various organs, including the liver, to form phospholipids (LDLs and HDLs), cholesterol and oxidized forms thereof. Oxidized phospholipids (OxPL) including OxLDL are known inflammatory mediators and strongly correlated with cardiovascular diseases. For example, Bieghs et al. (Hepatology, 65(3):894-903, 2012) describe that the use of antibodies to oxLDL led to a reduction in hepatic inflammation.

Increased amounts of adipose tissue are associated with decreased production of adiponectin. Data from studies in mice and humans increasingly implicate insufficient adiponectin as a major factor in the development of fatty liver and steatohepatitis. Adiponectin circulates as trimer (low molecular weight adiponectin), hexamer (medium molecular weight adiponectin) and higher order multimer (high molecular weight adiponectin) in serum and isoform-specific effects have been demonstrated. Epidemiological studies revealed that low adiponectin levels are associated with NASH. Moreover, adiponectin is believed to have a hepatoprotective effect due to protective effects against oxidative damage. Normal levels of adiponectin vary by age and sex. For example, females have a higher baseline adiponectin level compared to males. A normal weight female typically has an adiponectin level of between about 8.5 and 11 μg/ml and males typically have an adiponectin level of between about 6 and 8 μg/ml. In contrast, subject with fatty liver disease, NASH and/or obesity have adiponectin levels that are about 50-90% of normal levels (e.g., decreased by 10-50% from normal, or any value there between) (see, e.g., Merl et al. Int. J. Obes (Lond), 29(8), 998-1001, 2005).

In contrast, the resistin protein is increased in NASH subjects compared to normal subjects. Human resistin is a cysteine-rich, 108-amino-acid peptide hormone with a molecular weight of 12.5 kDa. In adult humans, resistin is expressed in bone marrow. Moreover, in adipocytes of subjects having a low or healthy BMI, resistin mRNA is almost undetectable. Consistent with this resistin concentrations in serum, and women may have higher resistin concentrations than men. Resistin mRNA expression in human peripheral mononuclear cells is increased by proinflammatory cytokines. Serum resistin is significantly elevated in both NASH and simple steatotic subjects. Hepatic resistin is significantly increased in NASH patients in both mRNA and protein levels than those in simple steatosis and normal control subjects. Because of the cysteine-rich structure of resistin changes in sulfur availability (mainly due to cysteine and glutathione) can have an effect on the protein's structure and function. As mentioned above, cysteamine and cystamine can modulated cysteine and/or glutathione levels in subjects taking cysteamine or cystamine.

Subjects afflicted with NAFLD or NASH tend to be in a higher percentile of weight for their age group (e.g., above the 97^{th} percentile for BMI for their age group). Treating pediatric patients at an early stage may have lifelong benefits in the management of liver function and obesity.

The compositions and methods of the disclosure demonstrate that deuterated compositions of the disclosure reduced liver fibrosis as well as improve liver function markers in animal models of NAFLD. For example, the data demonstrated that animal models treated with a high-fat NASH diet resulted in non-alcoholic steatohepatitis with fibrosis in the liver and that when administered a deuterated compound during the process to induce fatty liver, the deuterated compounds reduced the risk of developing NASH and markers thereof. In a further study, the disclosure demonstrates that following treatment to induce fatty liver disease administration of deuterated compounds of the disclosure resulted in an improvement in both ALT markers of liver function as well as a reduction in inflammatory infiltrate, liver fibrosis and markers of fibrosis such as collagen 1 and TIMP. Accordingly, the disclosure demonstrates that deuterated cystamine and/or cysteamine can be used to prevent and/or treat NAFLD, NASH and liver fibrosis resulting from these diseases.

The disclosure provides populations of subject with NASH that that have high probability of responding to treatment with a deuterated cysteamine or cystamine composition. The disclosure provides a method of treating a subject suffering from fatty liver disease, such as NASH, comprising administering a therapeutically effective amount of a compound of the disclosure. In one embodiment, the fatty liver disease is selected from the group consisting of non-alcoholic fatty acid liver disease (NAFLD), non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy.

In certain embodiments, pediatric and juvenile patients are treated with a deuterated compound of the disclosure. In one embodiment, a subject having NASH, biliary cholangitis, biliary atresia and the like is administered a formulation comprising a deuterated compound and/or prodrug of the disclosure for oral administration in an amount to obtain about 10-200 μmol (e.g., 10, 20, 30, 40, 50, 60, 70, 80, 90, 100, 110, 120, 130, 140, 150, 160, 170, 180, 190, 200, or any value there between) of a compound disclosed herein in the plasma. In a further embodiment, the formulation is a delayed release oral formulation. In one embodiment, the dose is about 10-95 mg/kg. In another embodiment, the dose is administered 2-4 times per day at about 100 mg to 1 gram per dose. Typically, the dose is changed over time to reach the highest tolerable dose for the subject, typically between about 30-200 μmol of the compound in plasma. For example, an initial dose may provide a circulating level of about 10 μmol of the compound, which will be adjusted up to the highest tolerable dose. In certain embodiments of any of the foregoing, subjects less than years of age (e.g., less than 14, 13, 12, 11, 10, 9, 8, 7, 6, 5, 4, 3, 2 or 1 years of age) and having a body mass index (BMI) above the 97^{th} percentile for their age are treated with a compound of the disclosure. In some embodiments, the subject has a BMI above 97^{th} percentile for the age and weighs less than 65 kg. In some embodiment, these same subjects have high triglyceride levels, low LDH, and low or low normal adiponectin levels. In still another embodiment, the subjects have high or high normal resistin levels. In various embodiments of any of the foregoing, the patient weighs less than 65 kg. In various embodiments, the patient weighs from about 35-65 kg, or from about 40-60 kg, or from about 45-55 kg, or about 35, 40, 45, 50, 55, 60 or 65 kg. In various embodiments, the patient weighing less than 65 kg receives 600 to 1200 mg/day of a deuterated compound of the disclosure or an amount to obtain circulating plasma levels of the compound of about 10-200 μmol (typically about 30-80 and more commonly about 40 μmol). In any of the foregoing embodiments, the subject has Type I NASH or NASH with Type 1 histological pattern. In still another embodiment of the foregoing the subject has lobular inflammation of the liver. In still another of further embodiment of any of the foregoing, the subject has low adiponectin and high triglycerides characteristic of NASH. In still another embodiment of any of the foregoing, the subject has a marker (e.g., AST, ALT, GGT or other liver marker) having a level consistent with NASH as described herein.

In various embodiments, the patient weighs 65-80 kg, and may receive 500 to about 2000 mg/day of a compound of the disclosure or an amount to obtain circulating plasma levels of the compound of about 10-200 μmol (typically about 30-80 and more commonly about 40 μmol). In any of the foregoing embodiments, the subject has Type I NASH. In still another embodiment of the foregoing the subject has lobular inflammation of the liver. In still another of further embodiment of any of the foregoing, the subject has low adiponectin and high triglycerides characteristic of NASH.

In various embodiments, the patient weighs more than 65 kg and receives 900 to about 2000 mg/day of a compound of the disclosure or an amount to obtain circulating plasma levels of the deuterated compound of about 10-200 μmol (typically about 30-50 and more commonly about 40 μmol). In any of the foregoing embodiments, the subject has Type I NASH. In still another embodiment of the foregoing the subject has lobular inflammation of the liver. In still another of further embodiment of any of the foregoing, the subject has low adiponectin and high triglycerides characteristic of NASH.

The subject can be an adult, adolescent or child. In various embodiments, the patient is from 2 to 7 years old, from 8 to 11 years old, from 9 to 12 years old, or from 13 to 18 years old. In various embodiments, an adolescent is from 10 to 19 years old as described in the National Institutes of Health standards.

In various embodiments, the administration results in a decrease in NAFLD Activity Score of two or more points, no worsening or an improvement of fibrosis, reduction in serum aminotransferases and gammaglutamyl transpeptidase (GGT); reduction in MRI-determined hepatic fat fraction; changes to markers of oxidation and anti-oxidant status; changes in fasting insulin and glucose; an increase in circulating adiponectin levels; a decrease in circulating resistin levels; a decrease in triglyceride levels; a decrease in oxidized phospholipids; changes in weight, height, body mass index (BMI) and waist circumference; changes in the Pediatric Quality of Life score; changes to any symptoms that patient may have experienced; proportion with a change from a histological diagnosis of definite NASH or indeterminate for NASH to not NASH at end of treatment; individual histological characteristics at end of treatment compared to baseline such as steatosis (fatty liver), lobular inflammation, portal chronic inflammation, ballooning, fibrosis score and stage 1a versus 1b fibrosis; and, change in mean NAS.

In various embodiments of the disclosure, a deuterated compound of the disclosure is administered at a daily dose ranging from about 10 mg/kg to about 2.5 g/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg or 1000 mg/kg, or may range between any two of the foregoing values. In some embodiments, the deuterated compound of the disclosure is administered at a total daily dose of from approximately 0.25 g/m² to 4.0 g/m² body surface area, about 0.5-2.0 g/m² body surface area, or 1-1.5 g/m² body surface area, or 1-1.95 g/m² body surface area, or 0.5-1 g/m² body surface area, or about 0.7-0.8 g/m² body surface area, or about 1.35 g/m² body surface area, or about 1.3 to about 1.95 grams/m²/day, or about 0.5 to about 1.5 grams/m²/day, or about 0.5 to about 1.0 grams/m²/day, e.g., at least about 0.3, 0.4, 0.5, 0.6, 0.7, 0.8, 0.9, 1.0, 1.1, 1.2, 1.3, 1.4, 1.5, 1.6, 1.7, 1.8, 1.9 or 2 g/m², or up to about 0.8, 0.9, 1.0, 1.1, 1.2, 1.3, 1.4, 1.5, 1.6, 1.7, 1.8, 1.9, 2.0, 2.2, 2.5, 2.7, 3.0, 3.25, 3.5 or 3.75 g/m² or may range between any two of the foregoing values.

In some embodiments, the delayed and extended-release formulation comprises an enteric coating that releases a compound disclosed herein when the formulation reaches the small intestine or a region of the gastrointestinal tract of a subject in which the pH is greater than about pH 4.5. In various embodiments, the formulation releases at a pH of about 4.5 to 6.5, 4.5 to 5.5, 5.5 to 6.5 or about pH 4.5, 5.0, 5.5, 6.0 or 6.5.

In a particular embodiment, the compound of the disclosure or a pharmaceutically acceptable salt, prodrug or solvate thereof is formulated for oral administration (e.g., as a capsule, table, caplet, solution, etc.). In a further embodiment, the disclosure provides for capsules, tablets, or caplets, comprising 50 mg to 200 mg of a compound of disclosure or a pharmaceutically acceptable salt (e.g., a bitartrate salt), prodrug or solvate thereof. In yet a further embodiment, the capsules, tablets, or caplets further comprise inactive ingredients, such as colloidal silicon dioxide, croscarmellose sodium, D&C yellow no. 10 aluminum lake, FD&C blue no. 1 aluminum lake, FD&C blue no. 2 aluminum lake, FD&C red no. 40 aluminum lake, gelatin, magnesium stearate, microcrystalline cellulose, pharmaceutical glaze, pregelatinized starch, silicon dioxide, sodium lauryl sulfate, synthetic black iron oxide and/or titanium dioxide.

In yet another embodiment, a compound disclose herein is administered at a frequency of 4 or less times per day (e.g., one, two or three times per day). In various embodiments, the composition is a delayed or controlled release dosage form that provides increased delivery of a compound disclosed herein to the small intestine.

In an embodiment, the compound of the disclosure or a pharmaceutically acceptable salt, prodrug or solvate thereof is formulated for oral administration (e.g., as a capsule, table, caplet, solution, etc.) that provides for delayed release. In a further embodiment, the disclosure provides for delayed release capsules, tablets, or caplets, comprising 25 mg to 75 mg of a deuterated compound of disclosure or a pharmaceutically acceptable salt (e.g., a bitartrate salt), prodrug or solvate thereof. In yet a further embodiment, the delayed release capsules, tablets, or caplets further comprise inactive ingredients, such as microcrystalline cellulose, Eudragit® L 30 D-55, Hypromellose, talc, triethyl citrate, sodium lauryl sulfate, purified water, gelatin, titanium dioxide, blue ink and/or white ink.

The delay or controlled release form can provide a Cₘₐₓ of a compound disclosed herein, or a biologically active metabolite thereof, that is at least about 35%, 50%, 75% or higher than the Cₘₐₓ provided by an immediate release dosage form containing the same amount of the compound. In another embodiment, the delay and extended release formulation provides an improved AUC compared to immediately release forms of the compound. For example, the AUC is increased compared to an immediate release formulation. In yet another embodiment, the delayed or controlled release dosage form comprises an enteric coating that releases a compound disclosed herein when the composition reaches the small intestine or a region of the gastrointestinal tract of a subject in which the pH is greater than about pH 4.5. In various embodiments, the pH is between 4.5 and 6.5. In one embodiment, the pH is about 5.5 to 6.5. In one embodiment the compound of the disclosure is delivered throughout the small intestine providing an extended release in the small intestine.

The delay or controlled release form can provide a Cₘₐₓ of a compound disclosed herein, or a biologically active metabolite thereof, that is at least about 10%, 20%, 30% or higher than the Cₘₐₓ provided by an enterically coated non-deuterated cystamine and/or cysteamine (e.g., Procysbi®) dosage form containing the same amount of the cysteamine and/or cystamine base. In another embodiment, the delay and extended-release formulation comprising a compound disclosed herein provides an improved AUC compared to approved formulations of cysteamine. For example, the AUC of a compound of the disclosure is increased compared to approved formulations of cysteamine. In yet another embodiment, the delayed or controlled release dosage form comprising a compound of the disclosure comprises an enteric coating that releases a compound disclosed herein when the composition reaches the small intestine or a region of the gastrointestinal tract of a subject in which the pH is greater than about pH 4.5. In various embodiments, the pH is between 4.5 and 6.5. In one embodiment, the pH is about 5.5 to 6.5. In one embodiment the compound of the disclosure is delivered throughout the small intestine providing an extended release in the small intestine.

In various embodiments, the enterically coated formulation comprising a compound of the disclosure is granulated and the granulation is compressed into a tablet or filled into a capsule. In certain embodiments, the granules are enterically coated prior to compressing into a tablet or capsule. Capsule materials may be either hard or soft, and are typically sealed, such as with gelatin bands or the like. Tablets and capsules for oral use will generally include one or more commonly used excipients as discussed herein.

A suitable pH-sensitive polymer is one which will dissolve in intestinal environment at a higher pH level (pH greater than 4.5), such as within the small intestine and therefore permit release of the pharmacologically active substance in the regions of the small intestine and not in the upper portion of the GI tract, such as the stomach.

In various embodiments, exemplary formulations comprising a deuterated compound of the disclosure that are contemplated for use in the present methods include those described in International Patent Applications PCT/US2007/002325, PCT/US2014/042607 and PCT/US2014/042616 (the disclosure of which are incorporated herein by reference).

For administration of the dosage form, i.e., the tablet or capsule comprising the enterically coated compound of the disclosure, a total weight in the range of approximately 50 mg to 1500 mg is used. In various embodiments, the tablet or capsule comprises 25, 50, 75, 100, 125, 150, 175, 200, 225, 250, 275, 300, 400 or 500 mg of the compounds of the disclosure as active ingredient, and multiple tablets or capsules are administered to reach the desired dosage. The dosage form is orally administered to a subject in need thereof.

In one embodiment, a tablet core comprises about 50 to 500 mg of a compound of the disclosure that is encapsulated in an enteric coating material having a thickness of about 60-100 μm (e.g., about 71, 73, 75, 77, or 79 μm or any value there between) and/or about 10-13% (e.g., about 10.5, 11.0, 11.2, 11.4, 11.6, 11.8, 12.0, 12.2, 12.4, 12.6, 12.8% of any value there between) by weight of the tablet. In another embodiment, a tablet core comprises about 100 to 100 mg of a compound of the disclosure about that is encapsulated in an enteric coating material having a thickness of about 90-130 μm (e.g., about 97, 99, 101, 103, 105, 107, 109, 111, 113 μm or any value there between) and/or about 9-14% (e.g., about 9.5, 9.7, 9.9, 10.1, 10.3 10.5, 11.0, 11.2, 11.4, 11.6, 11.8, 12.0, 12.2, 12.4, 12.6, 12.8, 13.0, 13.2, 13.4, 13.6, 13.8% or any value there between) by weight of the tablet by weight of the tablet.

In any of the foregoing embodiments, the enteric coating material can be selected from the group comprising polymerized gelatin, shellac, methacrylic acid copolymer type C NF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethyl cellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters.

The composition can be administered orally or parenterally. In another embodiment, the method results in improvement in liver fibrosis compared to levels before administration of the compound of the disclosure. In yet another embodiment, the method results in a reduction in fat content of liver, a reduction in the incidence of or progression of cirrhosis, or a reduction in the incidence of hepatocellular carcinoma. In one embodiment, the method results in a decrease in hepatic aminotransferase levels compared to levels before administration of the deuterated compound of the disclosure. In a further embodiment, the administering results in a reduction in hepatic transaminase of between approximately 10% to 70%, e.g. 10, 20, 30, 40, 50, 60 or 70% or any value between these numbers, compared to levels before treatment. In yet another embodiment, the administering results in a reduction in alanine or aspartate aminotransferase levels in a treated patient to approximately 50%, 40%, 30%, 20% or 10% above normal ALT levels, or at normal ALT levels. In yet other embodiment, the administering results in a reduction in serum ferritin levels compared to levels before treatment with a compound of the disclosure. In various embodiments, the administration results in a lowering of NAS score.

In any of foregoing embodiments, formulations for use in the methods described herein can comprise a pharmaceutically acceptable salt of the compound of the disclosure, such a chloride or bitartrate salt, instead of free base compound.

The methods and composition of the disclosure can also include administering a second agent in combination with a compound of the disclosure to treat a disease or disorder. Thus, in another embodiment of any of the foregoing methods or composition, the subject can be treated with a combination of active agents for treating cystinosis or fatty liver disorders, such as NAFLD and NASH. The combination includes a compound of the disclosure and one or more of metformin, statins, anti-oxidants, and/or antibodies against oxidized phospholipids. Such a combination can have unexpected synergy due to a multifaceted approach to modulating inflammation and inflammatory mediators. Such a combination would increase the anti-oxidant effects of adiponectin by increasing adiponectin levels, reduce triglyceride levels thereby reducing circulating phospholipids, reduce insulin resistance, and block the proinflammatory effects of oxidized phospholipids.

It is to be understood that while the disclosure has been described in conjunction with specific embodiments thereof, that the foregoing description as well as the examples which follow are intended to illustrate and not limit the scope of the disclosure. Other aspects, advantages and modifications within the scope of the disclosure will be apparent to those skilled in the art to which the disclosure.

### Examples

### Chemical Synthesis of New Prodrugs of Cysteamine and Cysteine:

### S-(2-((tert-butoxycarbonyl)amino)ethyl)-(S)-2-((tert-butoxycarbonyl)amino)-3-((4-methoxybenzyl)thio)propanethioate

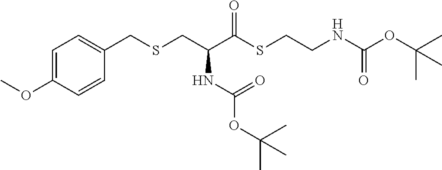 [SMILES: COc1ccc(CSC[C@H](NC(=O)OC(C)(C)C)C(=O)SCCNC(=O)OC(C)(C)C)cc1]

To a solution of N-(tert-butoxycarbonyl)-S-(4-methoxybenzyl)-L-cysteine (6.00 g, 1.00 Eq, 17.6 mmol) in THF (65.00 mL) cooled to 0° C., DCC (3.66 g, 1.01 Eq, 17.7 mmol) and DMAP (0.0215 g, 0.0100 Eq, 0.176 mmol) were added and the resulting mixture was stirred for 1 h. tert-Butyl(2-mercaptoethyl)carbamate (3.43 g, 1.1 Eq, 19.3 mmol) was then added at ambient temperature and stirring was continued for an additional 3 h. The reaction was then filtered and concentrated. Purification via silica gel chromatography (30% EtOAc in Hexanes) provided the title compound as a white solid (7.02 g, 14.0 mmol, 79.8%). ¹H NMR (600 MHz, Chloroform-d) δ 7.21 (d, J=8.6 Hz, 2H), 6.84 (d, J=8.6 Hz, 2H), 5.30 (d, J=8.5 Hz, 1H), 4.85 (s, 1H), 4.55-4.44 (m, 1H), 3.78 (s, 3H), 3.67-3.61 (m, 2H), 3.27 (t, J=6.7 Hz, 2H), 3.00 (t, J=7.0 Hz, 2H), 2.88-2.74 (m, 2H), 1.46 (s, 9H), 1.41 (s, 9H) ppm. HRMS (ES+) calculated for C₂₃H₃₆N₂O₆S₂[M+Na]⁺ 523.1907, found 523.1902.

S-(2-((tert-butoxycarbonyl)amino)ethyl)-(S)-2-((tert-butoxycarbonyl)amino)-3-((3-nitropyridin-2-yl)disulfaneyl)propanethioate

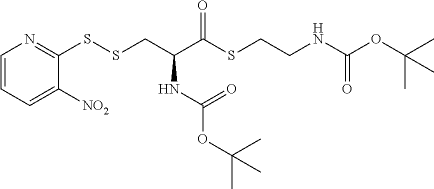 [SMILES: CC(C)(C)OC(=O)NCCSC(=O)[C@H](CSSc1ncccc1[N+](=O)[O-])NC(=O)OC(C)(C)C]

3-Nitropyridin-2-yl hypochlorothioite (0.476 g, 2.50 Eq, 2.50 mmol) was added to a solution of S-(2-((tert-butoxycarbonyl)amino)ethyl) (S)-2-((tertbutoxycarbonyl)amino)-3-((4-methoxybenzyl)thio)propanethioate (0.500 g, 1.00 Eq, 0.999 mmol) in DCM (20 mL) at 0° C. The resulting mixture was stirred for 2 h. Upon complete conversion of starting material as determined by TLC, the solvent was evaporated in vacuo. Purification via silica gel chromatography (DCM, 30% EtOAc in hexanes) provided the title compound as a yellow solid (0.254 g, 0.475 mmol, 47.6%). ¹H NMR (600 MHz, Chloroform-d) δ 8.95 (d, J=4.6 Hz, 1H), 8.53 (d, J=8.2 Hz, 1H), 7.40 (dd, J=8.3, 4.6 Hz, 1H), 7.08 (d, J=7.9 Hz, 1H), 4.87 (s, 1H), 4.57 (q, J=6.1 Hz, 1H), 3.57 (dd, J=14.0, 6.0 Hz, 1H), 3.23 (s, 3H), 3.13 (dd, J=14.0, 4.6 Hz, 1H), 2.99-2.92 (m, 3H), 1.46 (s, 9H), 1.39 (s, 9H) ppm. HRMS (ES+) calculated for C20H30N4O7S3 [M+Na]⁺ 557.1169, found 557.1164

### S-(2-((tert-butoxycarbonyl)amino)ethyl) (S)-2-((tert-butoxycarbonyl)amino)-3-((2-((tertbutoxycarbonyl) amino)ethyl)disulfaneyl)propanethioate

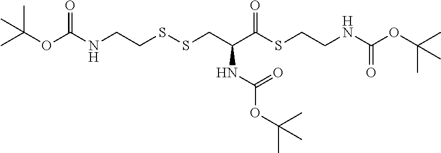 [SMILES: CC(C)(C)OC(=O)NCCSSC[C@H](NC(=O)OC(C)(C)C)C(=O)SCCNC(=O)OC(C)(C)C]

To a solution of S-(2-((tert-butoxycarbonyl)amino)ethyl) (S)-2-((tert-butoxycarbonyl)amino)-3-((3-nitropyridin-2-yl)disulfaneyl)propanethioate in ACN (0.325 mL) and water (0.425 mL) acidified to pH 6 by acetic acid, tert-butyl (2-mercaptoethyl)carbamate (0.040 g, 3 Eq, 0.22 mmol) was added at ambient temperature and the resulting mixture was stirred for 4 h. The reaction mixture was then diluted with water and extracted with EtOAc (3×). The combined organic layers were washed with saturated aqueous solution of NaCl, dried over anhydrous MgSO₄, filtered, and concentrated in vacuo. Purification via silica gel chromatography (30% EtOAc in hexanes) provided the title compound as a white solid (0.031 g, 0.056 mmol, 75%). ¹H NMR (600 MHz, Chloroform-d) δ 5.43 (d, J=8.5 Hz, 1H), 5.04 (s, 1H), 4.85 (s, 1H), 4.64 (q, J=7.4, 6.8 Hz, 1H), 3.44 (d, J=8.5 Hz, 2H), 3.31 (d, J=6.6 Hz, 2H), 3.13 (d, J=6.0 Hz, 2H), 3.04 (tq, J=13.6, 6.6 Hz, 2H), 2.86-2.77 (m, 2H), 1.49-1.41 (m, 27H) ppm. HRMS (ES+) calculated for C₂₂H₄₁N₃O₇S₃[M+Na]⁺ 578.1999, found 578.2005.
(S)-3-((2-ammonioethyl)disulfaneyl)-1-((2-ammonioethyl)thio)-1-oxopropan-2-aminium chloride. (BL-0856)

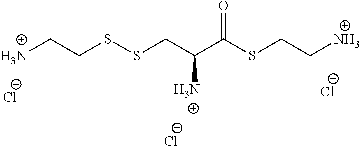 [SMILES: [NH3+]CCSSC[C@H]([NH3+])C(=O)SCC[NH3+] ; [Cl-] ; [Cl-] ; [Cl-]]

S-(2-((tert-butoxycarbonyl)amino)ethyl)-(S)-2-((tert-butoxycarbonyl)amino)-3-((2-((tertbutoxycarbonyl) amino)ethyl)disulfaneyl)propanethioate (25 mg, 1 Eq, 0.045 mmol) was dissolved in MeOH (0.600 mL) and 4M HCl in dioxane (0.300 mL, 27 Eq, 1.2 mmol) and the resulting mixture as stirred for 3 h at ambient temperature. The solvent was then evaporated in vacuo to afford the title compound as a white solid (16 mg, 0.044 mmol, 97%). ¹H NMR (600 MHz, Deuterium Oxide) δ 3.49 (dd, J=15.3, 4.8 Hz, 1H), 3.44 (t, J=6.6 Hz, 2H), 3.39 (q, J=6.5, 6.0 Hz, 2H), 3.36-3.32 (m, 1H), 3.32-3.21 (m, 2H), 3.15-3.02 (m, 2H) ppm. ¹³C NMR (150 MHz, Deuterium Oxide) δ 196.41, 58.64, 39.24, 38.24, 37.67, 33.86, 26.82 ppm. HRMS (ES+) calculated for C₇H₁₈N₃OS₃ [M+H]⁺ 256.0607, found 256.0603.

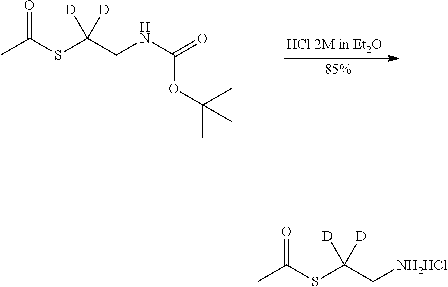

To a solution of thioester (0.110 g, 0.5 mmol, 1 equiv.) in diethyl ether (5 mL) at rt was added dropwise a solution of HCl (0.181 g, 2.49 mL, 4.97 mmol, 2 M, 10 equiv.) in diethylether. The solution was stirred at this temperature for 16 hours before it was concentrated. To this solid was then added cold diethyl ether and it was triturated and filtered over sintered glass to furnish the title compound (0.066 g, 0.42 mmol, 85%) as a white solid. ¹H NMR (600 MHz, D20): 3.22 (2H, s), 2.43 (3H, s) ppm. HRMS for C₄H₇D₂NOS calculated 121.0530 found 121.0528.

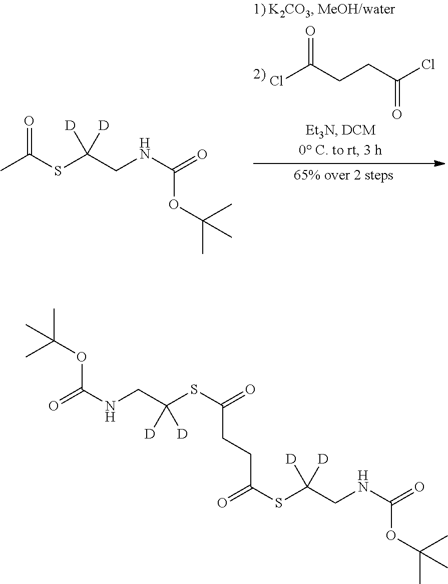 [SMILES: O=C(Cl)CCC(=O)Cl ; [2H]C([2H])(CNC(=O)OC(C)(C)C)SC(C)=O ; [2H]C([2H])(CNC(=O)OC(C)(C)C)SC(=O)CCC(=O)SC([2H])([2H])CNC(=O)OC(C)(C)C]

To a solution of thioester (0.378 g, 1.71 mmol, 1 equiv.) in a mixture of MeOH and water (1/1, 4 mL) was added K₂CO₃ (0.472 g, 3.42 mmol, 2 equiv.) and the reaction was stirred at rt for 1 hour before it was concentrated and extracted twice with EtOAc. The combined organic fractions were washed with brine twice, dried and concentrated under reduced pressure. The intermediate (0.246 mg, 1.37 mmol, 4 equiv.) was dissolved in dry DCM (4 mL) before triethylamine (0.070 g, 0.096 mL, 0.688 mmol, 2 equiv.) was added. The mixture was cooled to 0° C. before succinyl dichloride (0.053 g, 0.038 mL, 0.343 mmol, 1 equiv.) was added dropwise. The reaction was stirred for 2 hours at rt before it was concentrated and purified over flash chromatography (Hexanes/EtOAc:99/1 to 50/50) to furnish the title compound (0.129 g, 0.293 mmol, 65%) as a clear oil. ¹H NMR (600 MHz, CDCl₃): 4.43 (s, 2H), 3.26 (s, 4H), 2.75 (s, 4H), 1.43 (s, 18H) ppm.

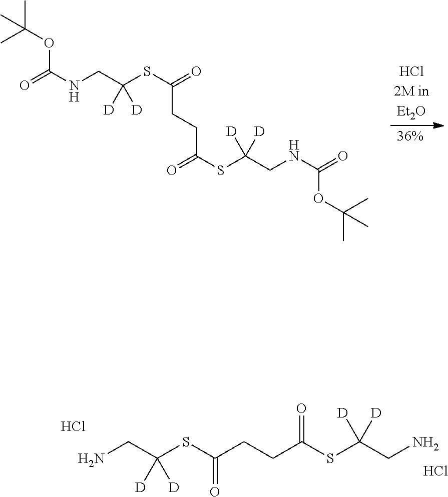 [SMILES: [2H]C([2H])(CNC(=O)OC(C)(C)C)SC(=O)CCC(=O)SC([2H])([2H])CNC(=O)OC(C)(C)C ; Cl ; [2H]C([2H])(CN)SC(=O)CCC(=O)SC([2H])([2H])CN ; Cl]

To a solution of thioester (0.060 g, 0.140 mmol, 1 equiv.) in diethyl ether (4 mL) at rt was added dropwise a solution of HCl (0.050 g, 0.680 mL, 1.400 mmol, 2 M, 10 equiv.) in diethylether. The solution was stirred at this temperature for 24 hours before it was concentrated. To this solid was then added cold diethyl ether and it was triturated and filtered over sintered glass to furnish the title compound (0.016 g, 0.051 mmol, 36%) as a white solid. ¹H NMR (600 MHz, D₂O): 3.21 (4H, s), 2.43 (4H, s) ppm. HRMS for C₈H₁₂D₄N₂O₂S₂ calculated 240.0904 found 240.0901.

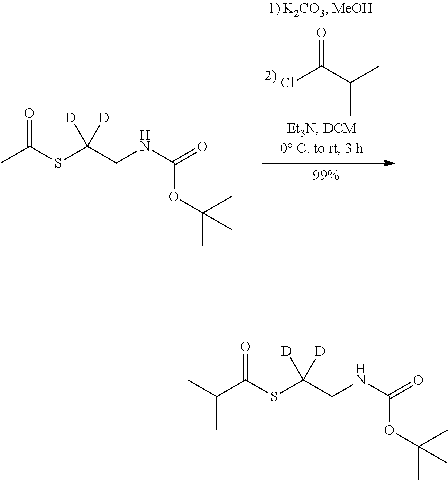

To a solution of thioester (0.348 g, 1.57 mmol, 1 equiv.) in a mixture of MeOH and water (1/1, 4 mL) was added K₂CO₃ (0.435 g, 3.14 mmol, 2 equiv.) and the reaction was stirred at rt for 1 hour before it was concentrated and extracted twice with EtOAc. The combined organic fractions were washed with brine twice, dried and concentrated under reduced pressure. The intermediate (0.307 mg, 1.73 mmol, 2 equiv.) was dissolved in dry DCM (2.5 mL) before triethylamine (0.088 g, 0.121 mL, 0.866 mmol, 1 equiv.) was added. The mixture was cooled to 0° C. before succinyl dichloride (0.093 g, 0.091 mL, 0.866 mmol, 1 equiv.) was added dropwise. The reaction was stirred for 2 hours at rt before it was concentrated and purified over flash chromatography (Hexanes/EtOAc:99/1 to 50/50) to furnish the title compound (0.214 g, 0.865 mmol, 99%) as a clear oil. ¹H NMR (600 MHz, CDCl₃): 4.47 (s, 1H), 3.29 (s, 2H), 2.77-2.72 (m, 1H), 1.43 (s, 9H), 1.19 (d, J=6 Hz, 6H) ppm.

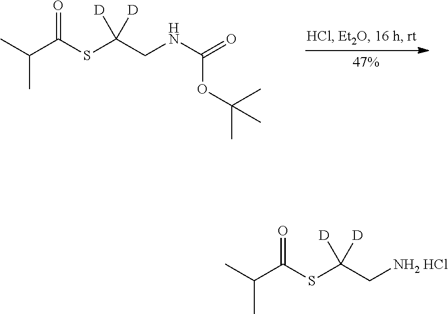

To a solution of thioester (0.220 g, 0.882 mmol, 1 equiv.) in diethyl ether (4 mL) at rt was added dropwise a solution of HCl (0.161 g, 2.210 mL, 4.410 mmol, 2 M, 5 equiv.) in diethylether. The solution was stirred at this temperature for 24 hours before it was concentrated. To this solid was then added cold diethyl ether and it was triturated and filtered over sintered glass to furnish the title compound (0.075 g, 0.040 mmol, 46%) as a white solid. ¹H NMR (600 MHz, D₂O): 3.21 (s, 2H), 2.93-2.96 (m, 1H), 1.19 (d, J=6 Hz, 6H) ppm. HRMS for C₆H₁₁D₂NOS: calculated 149.0843 found 149.0842.

Drug concentrations: BL-0856 powder was resuspended in water at a concentration of 25 mM. 40 μL of this solution was added to 10 mL of media for a final concentration of 100 uM. Some of the resuspended drug contained cysteamine and cystamine after testing 1-week post-resuspension.

Cystamine was made from high pH oxidation of cysteamine-bitartrate powder (w/Ammonium hydroxide), at a concentration of 25 mM. Nearly 25% of drug remained as cysteamine during time of experiment, and nearly 75% was oxidized to cystamine. Since this was used for only several timepoints of a control, this was deemed sufficient for this purpose. The drug was also administered to cells at a final concentration of 100 uM.

Based upon the LC-MS profile, BL-0856 was likely >80-90% pure (unhydrolyzed) when used in the experiments. A method for the reduced intermediate form was also made, but none of it was found present in the neat standard.

In vitro experiments with prodrugs in cystinotic fibroblasts. Fibroblasts from a de-identified cystinotic patient were split 3× from 2 plates into 12 plates. An experimental compound, BL-0856, was studied using the following protocols. Several timepoints of cystamine was also performed for comparison. The constant incubation time series for the new compound was: 0, 15 min, 30 min, 1 h, 3 h, and 5 h. The cystamine timepoints were 0, 30 min, 1 h, and 5 h. There was also a washout experiment done for 2 timepoints of the experimental compound, where after 3 hours of constant incubation, the media was exchanged with no compound, and incubated for either 1 h or 3 h. Harvesting of cells on the plate went as follows 2× washes with PBS that included 5 mM N-ethylmaleimide for trapping free thiol groups, and then scraping the plates with an acidified organic solvent extraction solution containing d₄-NEM-cysteamine, d₄-cystine, d₈-cystamine, ¹³C¹⁵N NEM-glutathione, and ¹³C, ¹⁵N GSSG as internal standards. All samples were run on an API 4500 triple quadrupole LC-MS/MS, with selective reaction monitoring (SRM/MRM) for the following compounds of interest: cystine, cysteine, cystamine, reduced glutathione, oxidized glutathione (GSSG). BL-0856 and the thiol reduced intermediate were scanned for, but were not found at high enough levels to be identified in any of the timepoints, likely due to a near immediate reduction and/or hydrolysis of the drug in the cell. All data fit well to single exponential curves (either decay or association), except where mentioned.

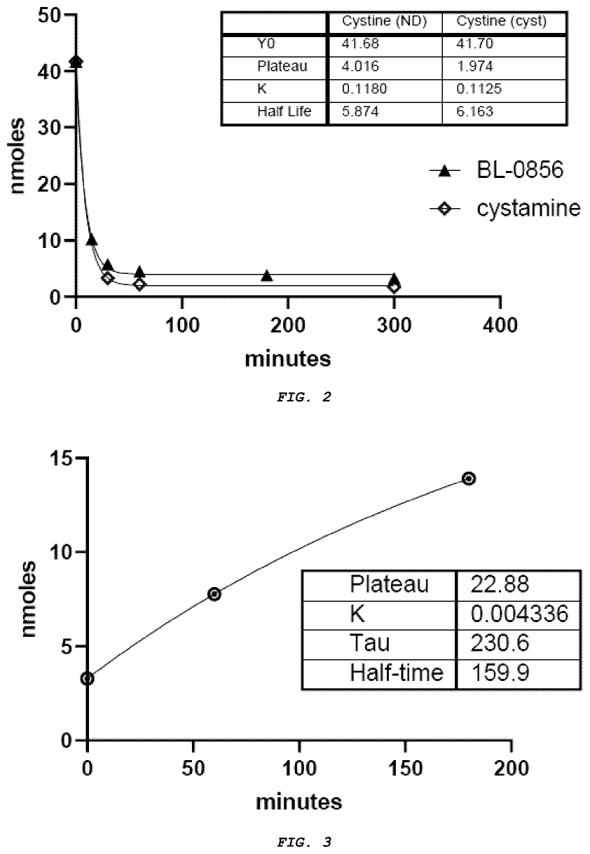

Cystine measurement during constant cystamine or BL-0856 exposure: Fibroblast cultures are exposed to 100 uM cystamine or 100 uM BL-0856 for the following timepoints: 0 min, 10 min, 30 min, 1 h, 3 h, and 5 h. Following incubation, cells are washed 2× in PBS containing N-ethylmaleimide. After removing the wash, the cells are harvested on ice using 80% acetonitrile/1% formic acid, containing stable isotope internal standard for cystine (d₄-cystine). As shown in FIG. 2, the difference in cystine depletion rates were nearly identical between cystamine and BL-0856.

Cystine measurement after washout: Fibroblast cultures are exposed to 100 uM BL-0856 for 3 h. Following incubation, cells are washed with PBS, and then incubated with regular media (containing FBS) for the following timepoints: 0 min, 1 h, and 3 h. Cells are harvested identically as described previously. The cystine reaccumulation rate for BL-0856 (see FIG. 3) was comparable to cystamine (data not shown).

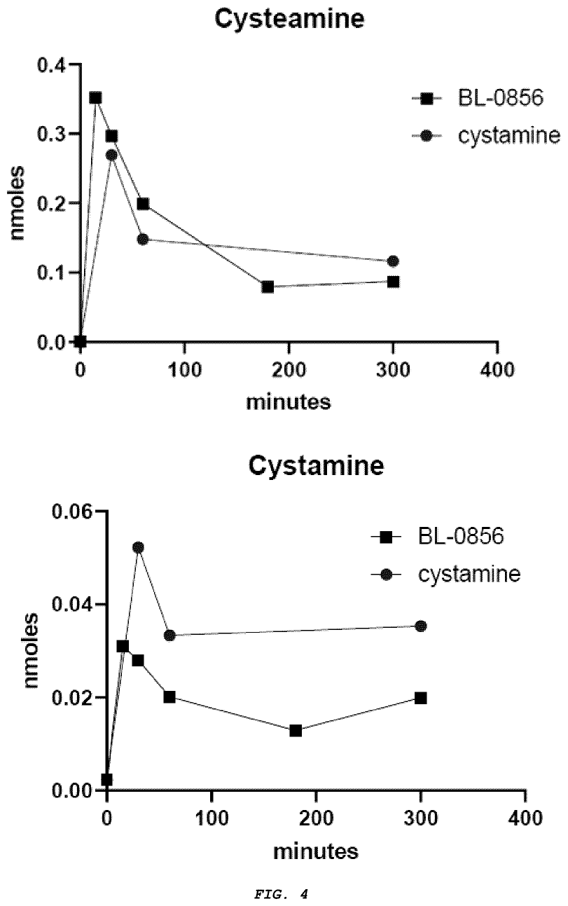

Cysteamine and cystamine measurement during constant cystamine or BL-0856 exposure: Fibroblast cultures are exposed to 100 uM cystamine or 100 uM BL-0856 for the following timepoints: 0 min, 10 min, 30 min, 1 h, 3 h, and 5 h. Following incubation, cells are washed 2× in PBS containing N-ethylmaleimide. After removing the wash, the cells are harvested on ice using 80% acetonitrile/1% formic acid, containing stable isotope internal standard for cystamine or cysteamine. As shown in FIG. 4, cystamine levels are higher in cystamine treated, yet some cystamine still exists in BL-0856 treated cells. Cysteamine levels are nearly identical in both treatments, indicating that reduction of cysteamine from disulfide precursors is rapid in both.

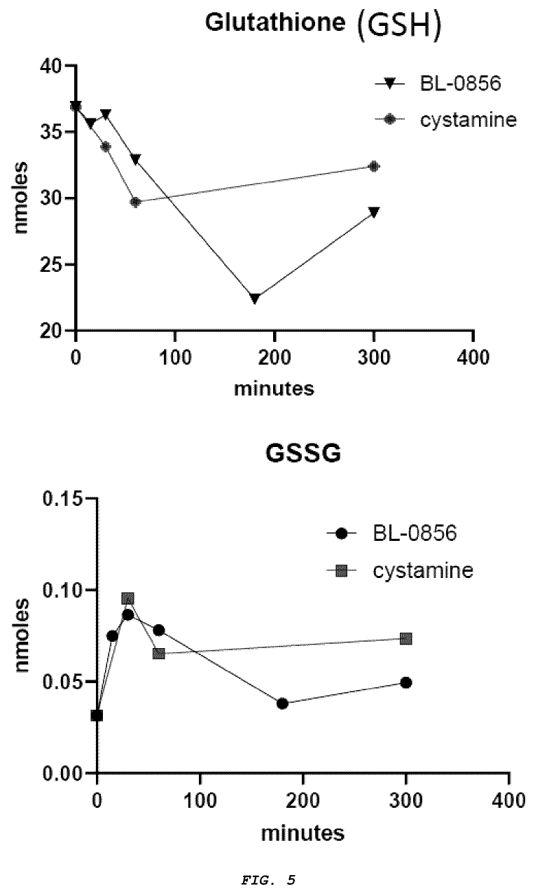

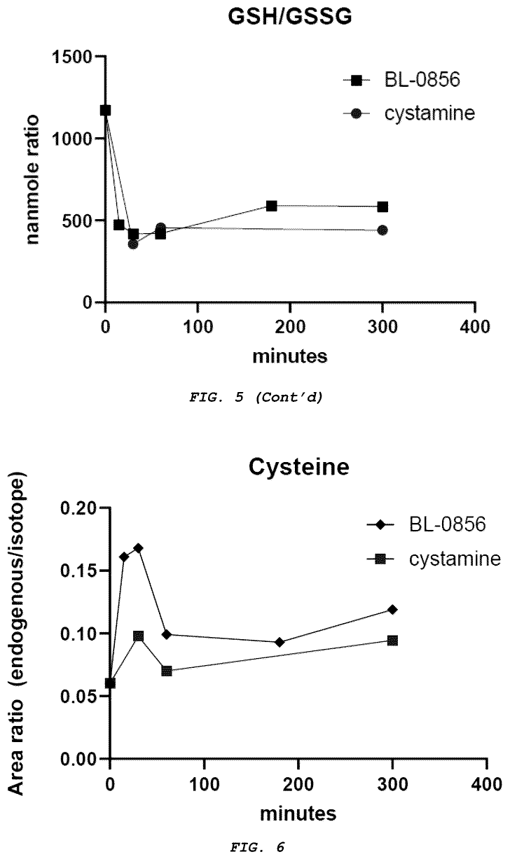

Glutathione and oxidized glutathione measurement during constant cystamine or BL-0856 exposure: Fibroblast cultures are exposed to 100 uM cystamine or 100 uM BL-0856 for the following timepoints: 0 min, 10 min, 30 min, 1 h, 3 h, and 5 h. Following incubation, cells are washed 2× in PBS containing N-ethylmaleimide. After removing the wash, the cells are harvested on ice using 80% acetonitrile/1% formic acid, containing stable isotope internal standard for glutathione and oxidized glutathione. As shown in FIG. 5, there was no obvious differences in glutathione between 2 drug forms. Of interest is that with both drugs, oxidized glutathione (GSSG) goes up at the first timepoint, and ratio of GSH/GSSG is lower throughout all timepoints in comparison to the 0-minute controls.

Cysteine measurement during constant cystamine or BL-0856 exposure: Fibroblast cultures are exposed to 100 uM cystamine or 100 uM BL-0856 for the following timepoints: 0 min, 10 min, 30 min, 1 h, 3 h, and 5 h. Following incubation, cells are washed 2× in PBS containing N-ethylmaleimide. After removing the wash, the cells are harvested on ice using 80% acetonitrile/1% formic acid, containing stable isotope internal standard for glutathione and oxidized glutathione. As shown in FIG. 6, cysteine is higher in the BL-0856 treated cells, indicating that cysteine is being released from the precursor form.

Results. BL-0856 is broken down early in the cell, as evident by early release of both cysteamine and cysteine. Cystine depletion and reaccumulation kinetics appear similar to cystamine. There is no evidence of increased glutathione accumulation, but the reduced/oxidized levels of glutathione go down after both drugs are administered.

Cystine Depletion Study. Dermal cystinotic fibroblasts were cultured in medium containing BL0948, BL0940 or D4-Cystamine at the same 100 uM concentration:

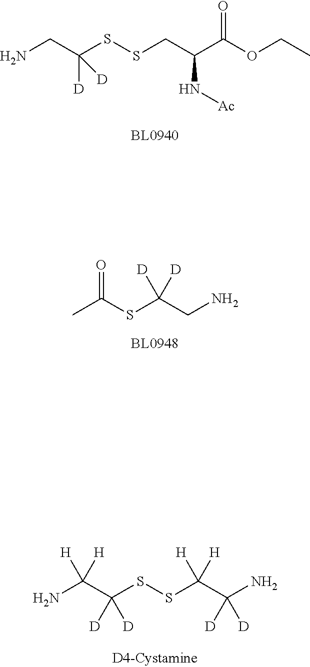

BL0984 and BL0940 will yield one molecule of D2 cysteamine whereas D4 Cystamine will yield 2 molecules of D2 cysteamine. The fact that BL-940 produces less of an effect compared to D4-cystamine is expected based on the fact that if dosed equally (mg/kg) as D4-cystamine, and assuming complete prodrug activation, the molecule would have half of the D2-cysteamine in the BL-940 treated cells compared to the D4-cystamine treated cells. An unexpected result is that BL-948 produced a pronounced effect considering that it does not convert entirely to D2-cysteamine as a significant portion of the prodrug spontaneously rearranges to the corresponding N-acetylated isomer that is more stable. This is interesting because it would indicate that N-acetyl-D2-cysteamine is more potent than D2-cysteamine in depleting cystine.

In the study, the cells are incubated in 100 uM of drug, therefore, ample drug is available in both free cysteamine and rearranged isomer forms.

BL-0940 D2-cysteamine joins to N-acetyl cysteine which is a mutual prodrug and stable. The non-deuterated compound would be known.

This prodrug analog, BL0940, is very stable both in the medium and within the cell.

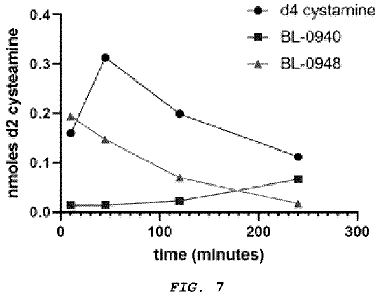

Intracellular D2-cysteamine levels are measured following continual drug exposure (in media) to cystinotic fibroblasts (FIG. 7).

Intracellular cystine depletion over time. The reduction in cystine levels following exposure to D4-cystamine and BL0948 is rapid and more profound. This likely represents a rapid exposure to D2-cysteamine after intracellular uptake. D4-cystamine will most likely reduce after crossing the cell membrane (peak level seen at 45 mins). BL0948 most likely has reduced to D2-cysteamine and the rearranged isomer in the medium before crossing the cell membrane. It is possible that after extracellular reduction that the ratio of D2-Cysteamine to rearranged analog is 10-20:1. This is why a relatively high level of intracellular D2-cysteamine at baseline is observed with BL0948 and why a dramatic reduction of cystine level is observed.

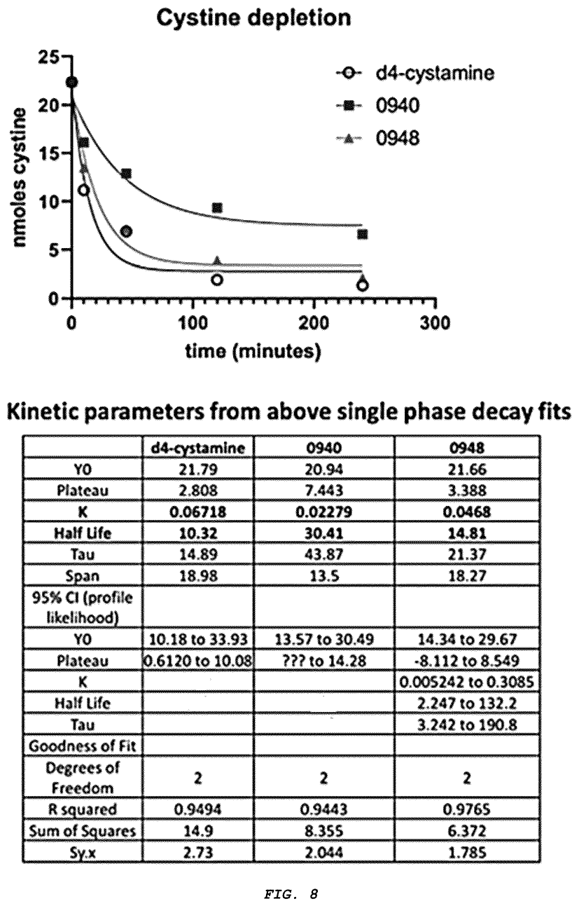

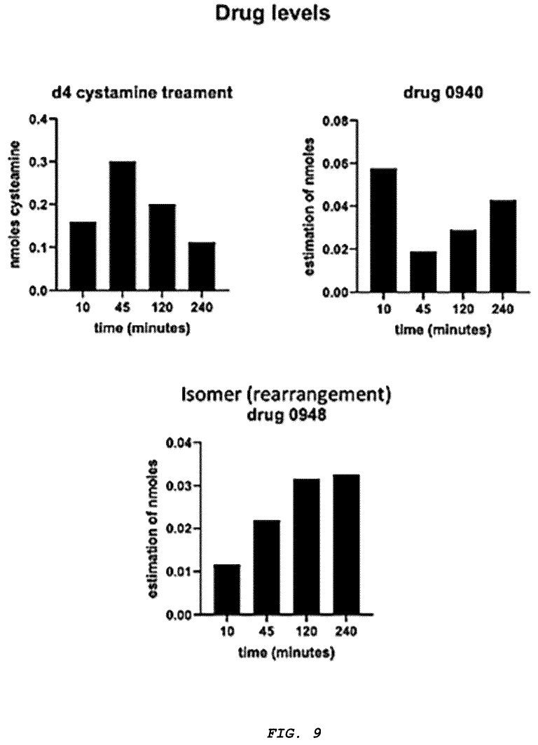

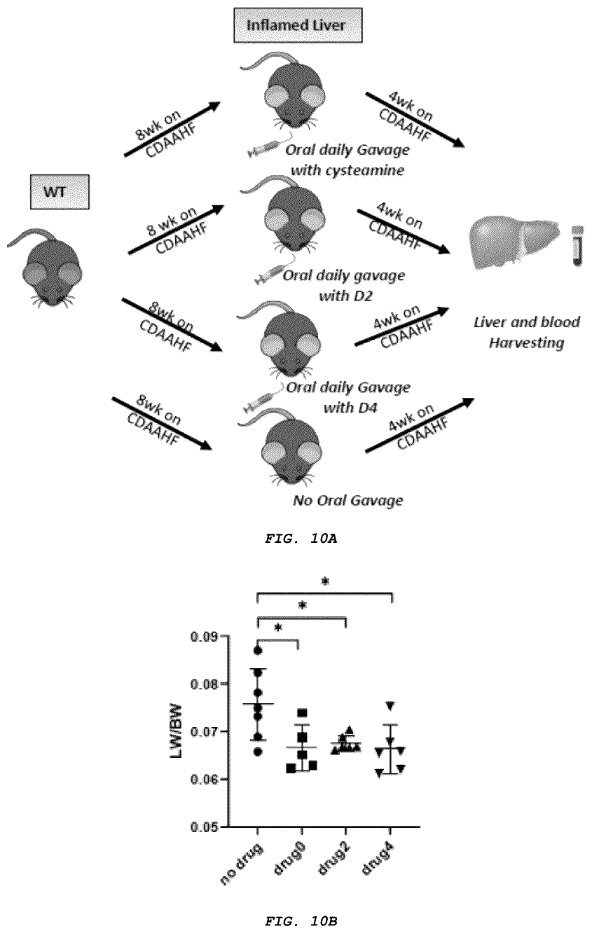

As for BL0940 it is possible that the disulfide bond in this molecule breaks less rapidly and therefore may provide a slower, but, longer lasting effect. Of note, following BL0940 continued exposure, the intracellular levels of BL0940 and D2-cysteamine increase over time (e.g., at 240 minute time points) (FIGS. 8-9).

### Studies in Choline-Deficient, L-Amino Acid-Defined, High-Fat Diet (CDAHFD) Induced Mouse Models of Non-Alcoholic Statohepatitis (NASH).

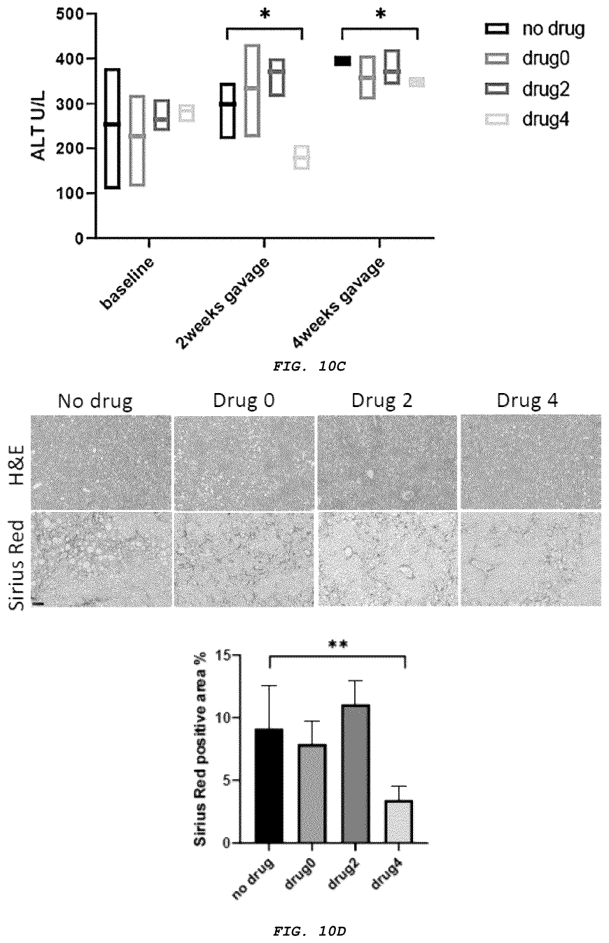

Study 1—NASH/Mouse. Mice had at least received 8 weeks of CDAHFD diet before starting the study and continued to receive this diet during the next phase of the study. There were 4 mice per group (C57BL/6 male):
Control group (c1), water administered once daily by gavage for 2 weeks;
200 mg/kg of Drug 0 once daily gavage therapy for 2 weeks;
200 mg/kg of Drug 2 once daily gavage therapy for 2 weeks;
200 mg/kg of Drug 4 once daily gavage therapy for 2 weeks.
   FIG. 10A depicts the study outline.

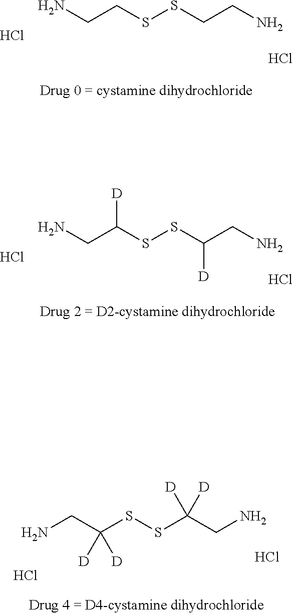

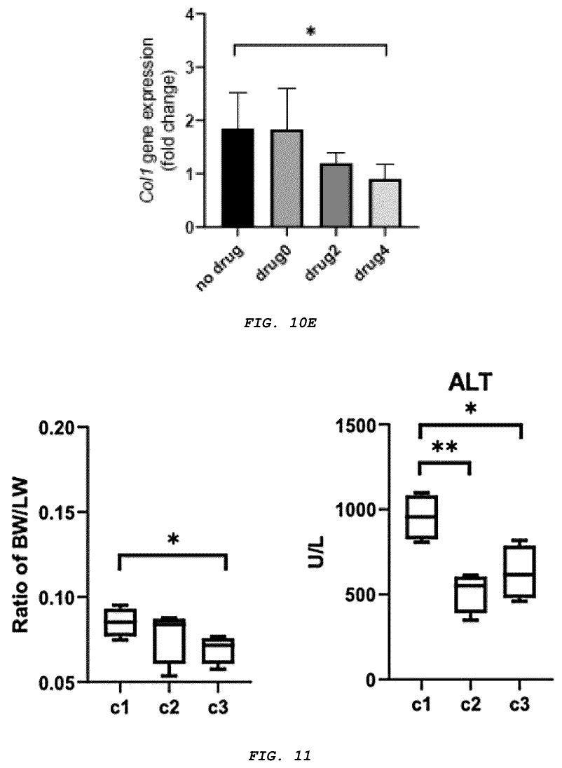

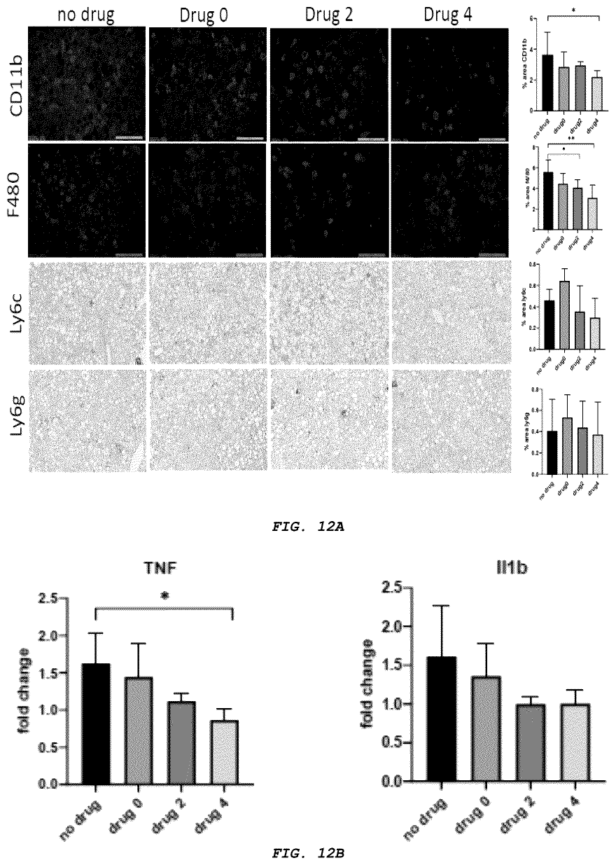

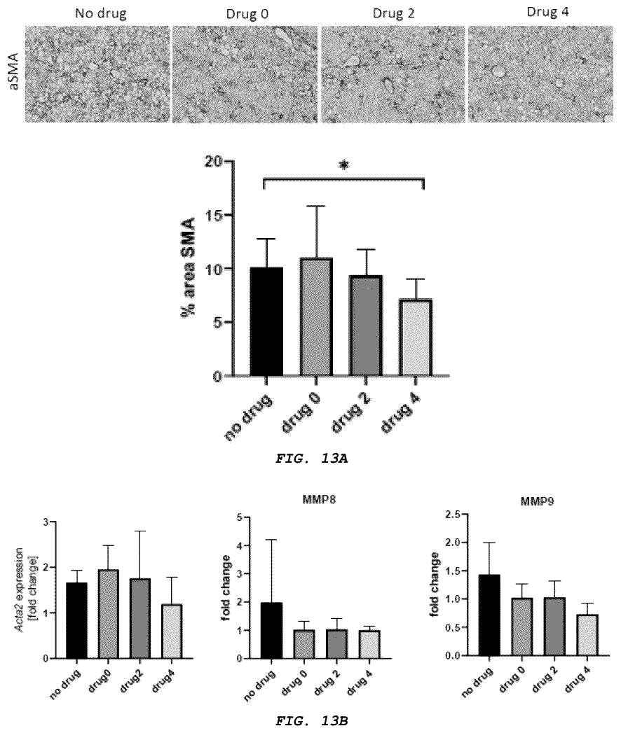

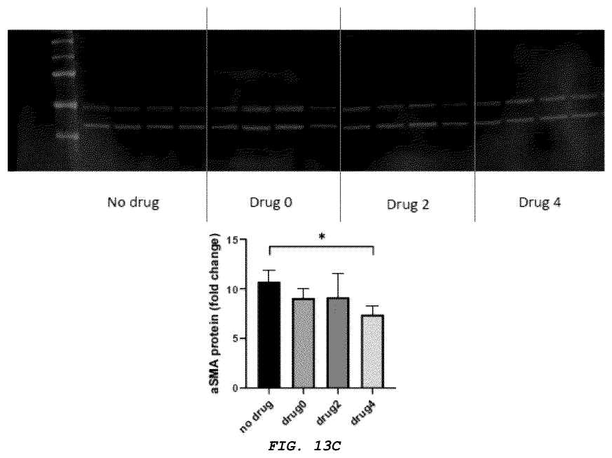

FIG. 10B-E shows a results of study 1 Hepatic tissue after 10 weeks of continued CDAHFD diet showing red staining of intrahepatic fibrosis. In addition, the treated group showed a significant reduction in hepatic transaminase ALT compared to control (FIG. 11). A significant reduction in intrahepatic fibrosis is noted with D2 and D4 compared with control group. FIG. 12A-B further shows a significant reduction in markers of inflammation when comparing D2-cystamine and D4-cystamine with the control groups. A significant reduction in markers of inflammation (F4/80) and fibrosis (aSMA) was noted when comparing D2-Cystamine and D4-Cystamine with the control groups (FIG. 13A-C).

### Murine CDAHFD NASH Study 2—Comparing Two Prodrug Analogs BL0940 and BL0948.

Mice had at least received 8 weeks of CDAHDF diet before starting the study and continued to receive this diet during the next phase of the study. There were 8 mice per group (C57BL/6 male).

### Control Group

HD BL0940 (High dose) once daily by gavage therapy for 4
LD BL0940 (low dose) once daily by gavage therapy for 4
BL0948 once daily by gavage therapy for 4 weeks

The MW of BL0940 is higher than BL0948 and so a higher dose was given. That is, HD BL0940 and BL0948 had capability of releasing same number of D2-cysteamine molecules.

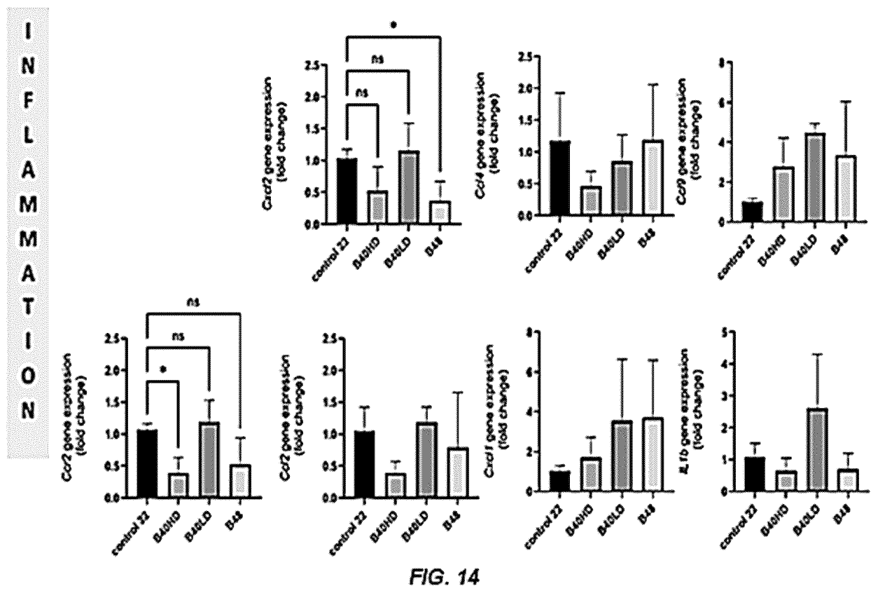

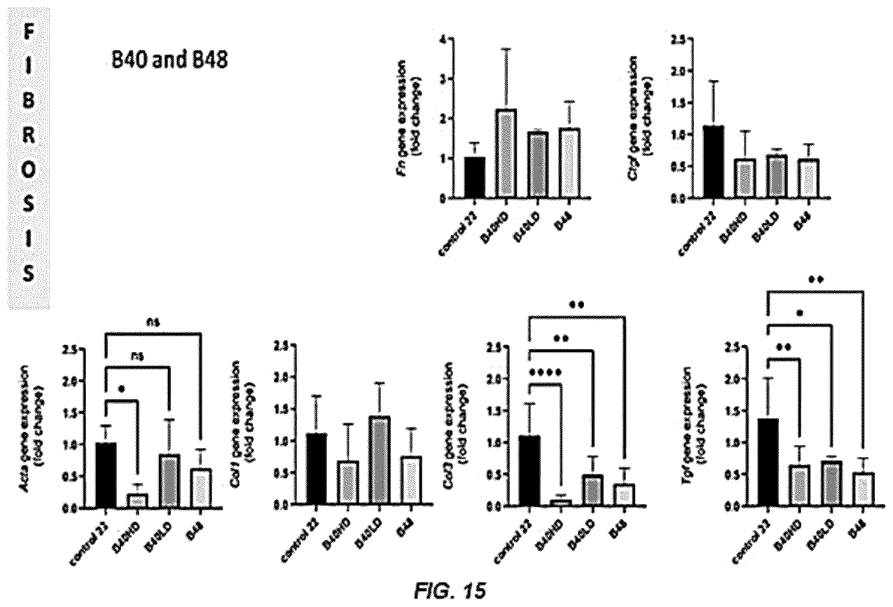

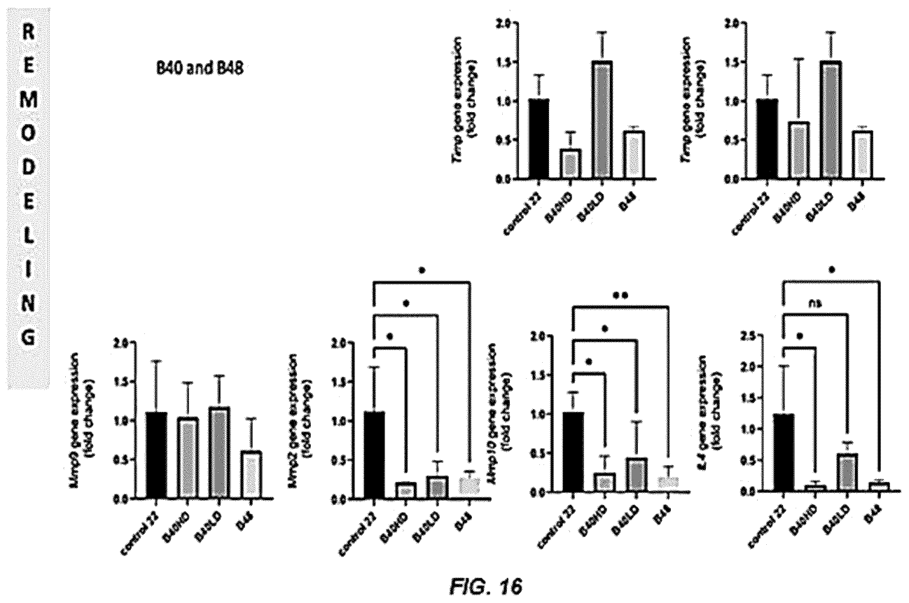

FIGS. 14-16 shows the results of the prodrug study and demonstrates a reduction in various markers of inflammation and disease. In the mouse studies of NASH (using the CDAHFD model) the higher dose of BL0940, and the same molar doses of BL0948 and D4 seem to bring about a significant effect on markers of inflammation, remodeling and fibrosis. However, and not surprisingly, the lower dose of BL0940 (which would release less D2 cysteamine) did not have the same degree of effect in the NASH mouse studies.

It will be understood that various modifications may be made without departing from the spirit and scope of this disclosure. Accordingly, other embodiments are within the scope of the following claims.

1. A compound having the structure of Formula I: 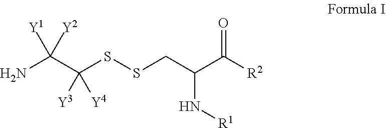
or a pharmaceutically acceptable salt or solvate thereof, wherein:
R¹ is selected from H or an acetyl group;
R² is selected from 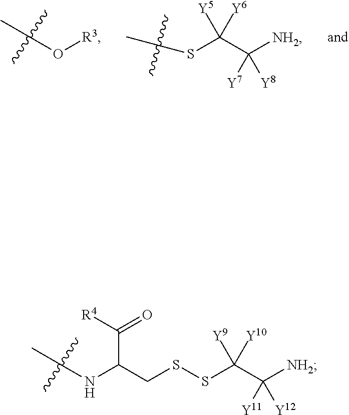
R³ is selected from an optionally substituted (C₁-C₆)alkyl, an optionally substituted cycloalkyl, an optionally substituted benzyl, or an optionally substituted aryl;
R⁴ is selected from 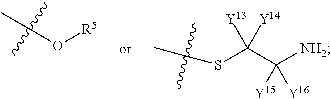
and
R⁵ is selected from an optionally substituted (C₁-C₆)alkyl, an optionally substituted cycloalkyl, an optionally substituted benzyl, or an optionally substituted aryl; and
Y¹-Y¹⁶ are each independently selected from H or D.

2. The compound of claim 1, wherein at least one of Y¹-Y¹⁶ independently has deuterium enrichment of no less than about 10%.

3-5. (canceled)

6. The compound of claim 1, wherein the compound has a structural formula of Formula II(a): 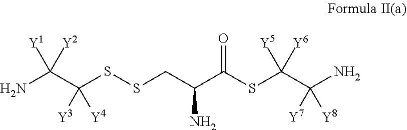
or a pharmaceutically acceptable salt or solvate thereof, wherein:
Y¹-Y⁸ are each independently selected from H or D.

7. The compound of claim 6, wherein the compound has a structural formula selected from: 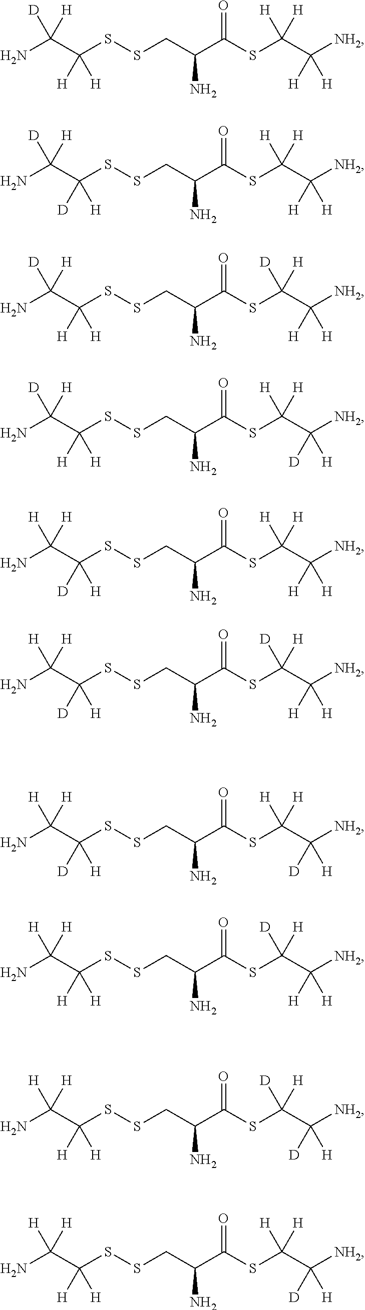 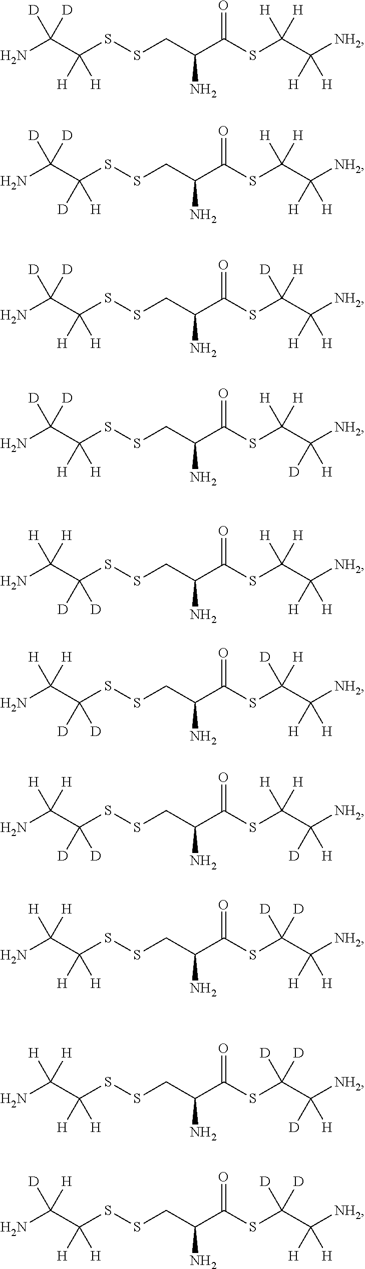 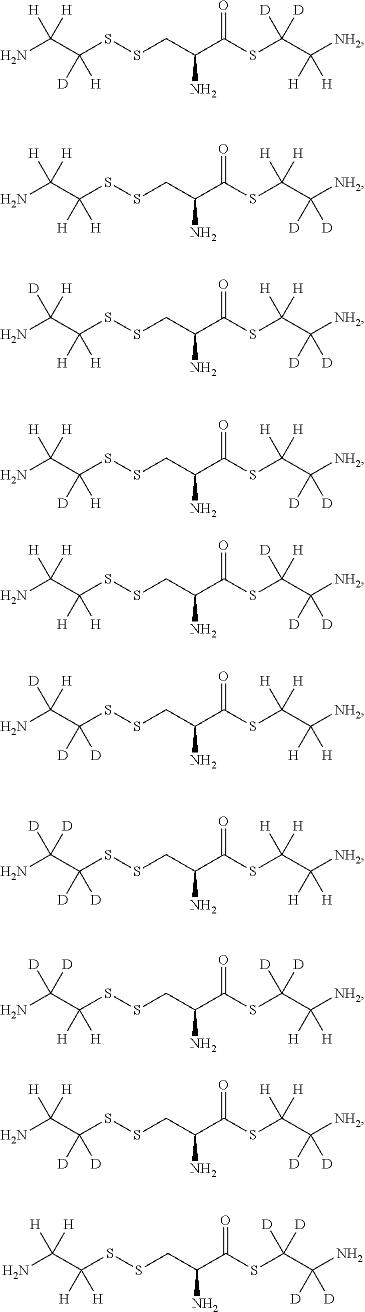 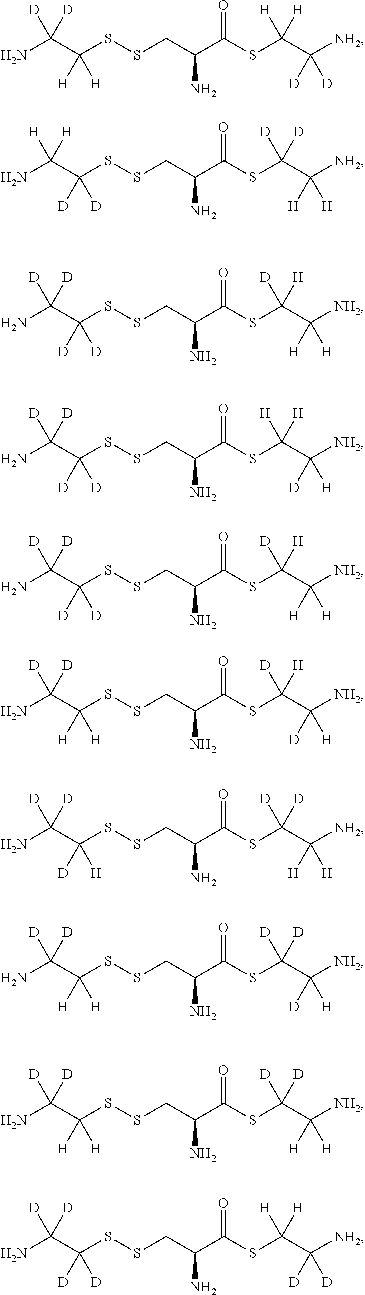 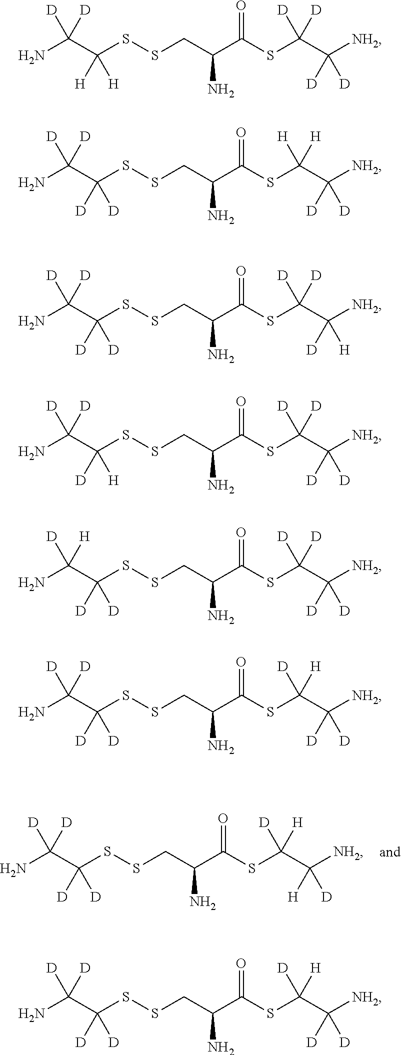
a pharmaceutically acceptable salt or solvate of any one of the foregoing.

8. The compound of claim 1, wherein the compound has a structure of: 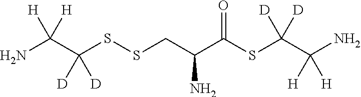 [SMILES: [2H]C([2H])(CN)SSC[C@H](N)C(=O)SC([2H])([2H])CN]
or a pharmaceutically acceptable salt, solvate, or prodrug thereof.

9. The compound of claim 6, wherein the pharmaceutically acceptable salt has the structure of Formula II(b): 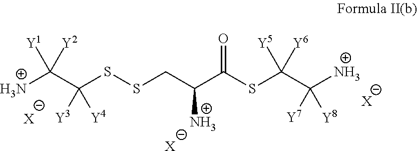
wherein,
Y¹-Y⁸ are each independently selected from H or D; and
X is a pharmaceutically acceptable counter ion.

10. The compound of claim 9, wherein the pharmaceutically acceptable counter ion is a bitartrate ion or chloride ion.

11. The compound of claim 9, wherein the compound has the structure of

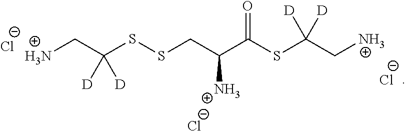 [SMILES: [Cl-] ; [2H]C([2H])(C[NH3+])SSC[C@H]([NH3+])C(=O)SC([2H])([2H])C[NH3+] ; [Cl-] ; [Cl-]]

12. The compound of claim 1, wherein the compound has the structure of Formula II(c): 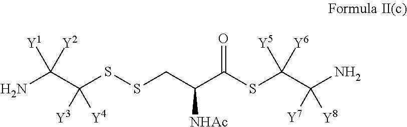
or a pharmaceutically acceptable salt or solvate thereof, wherein:
Y¹-Y⁸ are each independently selected from H or D; and
Ac refers to an acetyl group.

13. The compound of claim 12, wherein the compound has a structural formula selected from: 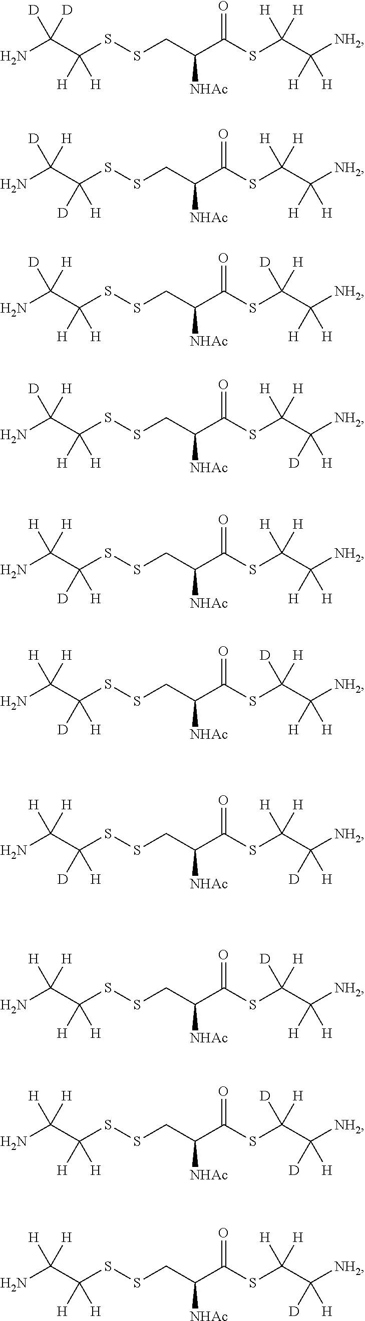 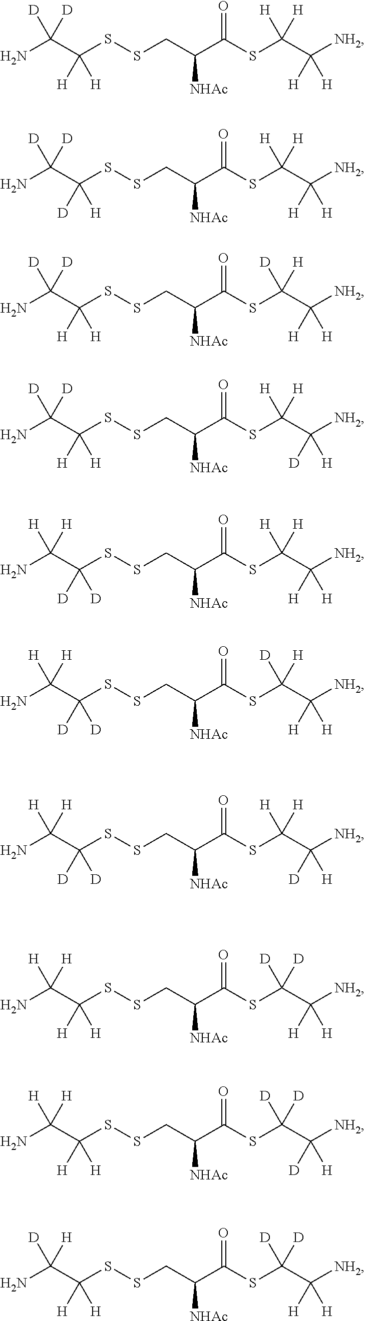 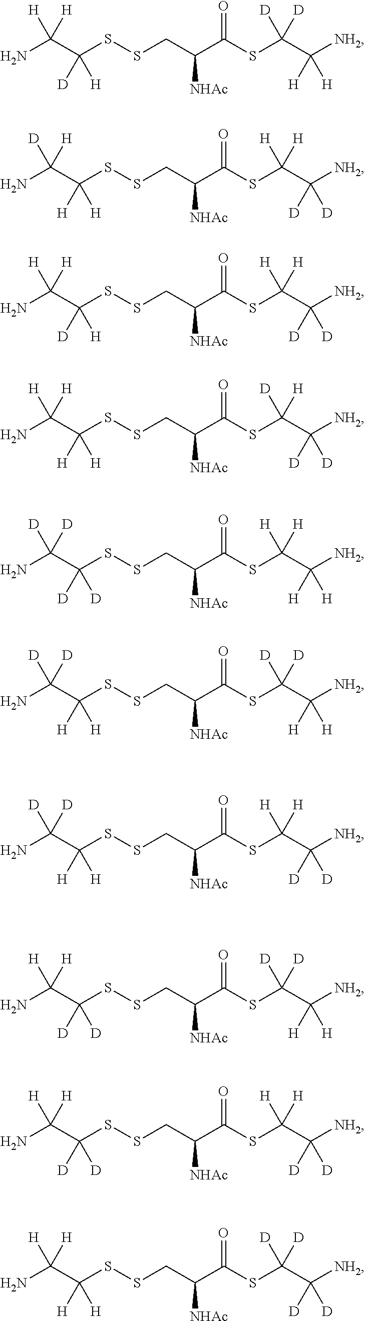 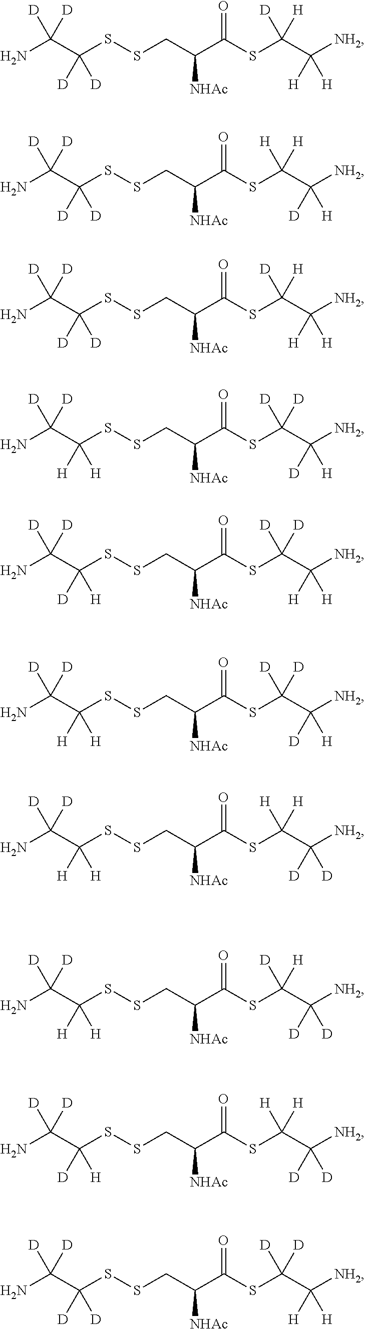 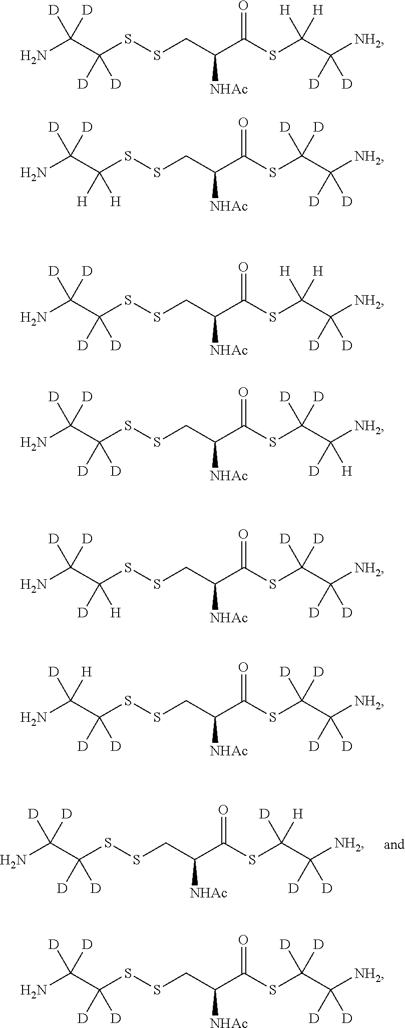
a pharmaceutically acceptable salt or prodrug thereof.

14. The compound of claim 12, wherein the pharmaceutically acceptable salt has the structure of Formula II(d): 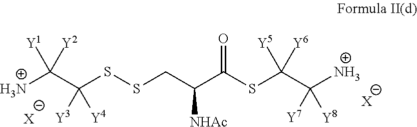
wherein,
Y¹-Y⁸ are each independently selected from H or D;
Ac is an acetyl group; and
X is a pharmaceutically acceptable counter ion.

15. The compound of claim 14, wherein the pharmaceutically acceptable counter ion is a bitartrate ion or chloride ion.

16. The compound of claim 14, wherein the compound has the structure of:

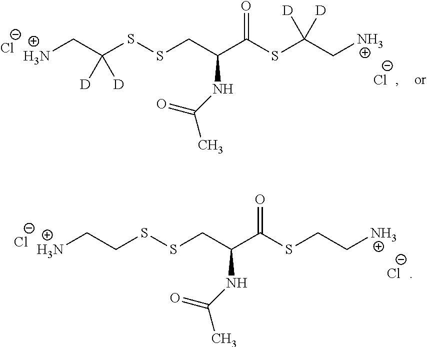 [SMILES: [Cl-] ; [2H]C([2H])(C[NH3+])SSC[C@H](NC(C)=O)C(=O)SC([2H])([2H])C[NH3+] ; [Cl-] ; [Cl-] ; CC(=O)N[C@@H](CSSCC[NH3+])C(=O)SCC[NH3+] ; [Cl-]]

17. The compound of claim 1, wherein the compound has a structural formula of Formula III(a): 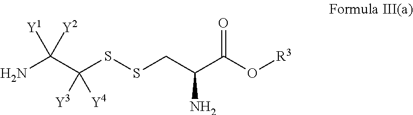
or a pharmaceutically acceptable salt or solvate thereof, wherein:
Y¹-Y⁴ are each independently selected from H or D; and
R³ is selected from a (C₁-C₆)alkyl.

18. The compound of claim 17, wherein the compound has a structural formula selected from: 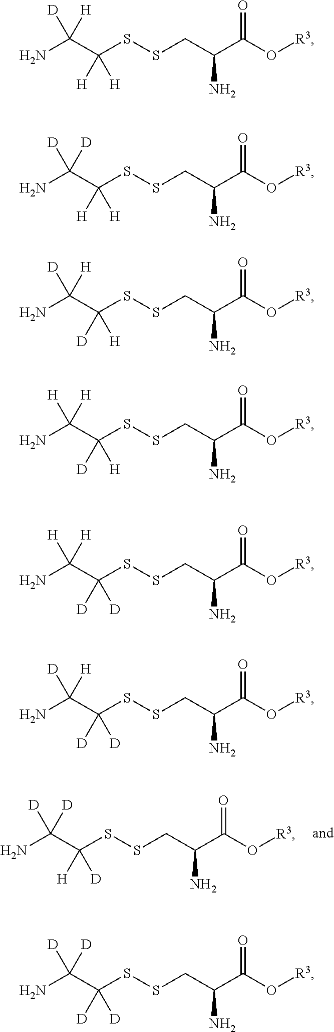
or a pharmaceutically acceptable salt or solvate of any one of the foregoing, wherein R³ is a (C₁-C₆)alkyl.

19. The compound of claim 17, wherein the pharmaceutically acceptable salt has the structure of Formula III(b): 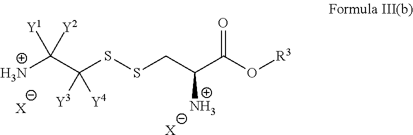
wherein,
Y¹-Y⁴ are each independently selected from H or D;
R³ is a (C₁-C₆)alkyl; and
X is a pharmaceutically acceptable counter ion.

20. The compound of claim 1, wherein the compound has the structure of Formula III(c): 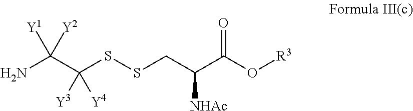
or a pharmaceutically acceptable salt or solvate thereof, wherein:
Y¹-Y⁴ are each independently selected from H or D; and
R³ is a (C₁-C₆)alkyl.

21. The compound of claim 20, wherein the compound has a structural formula selected from: 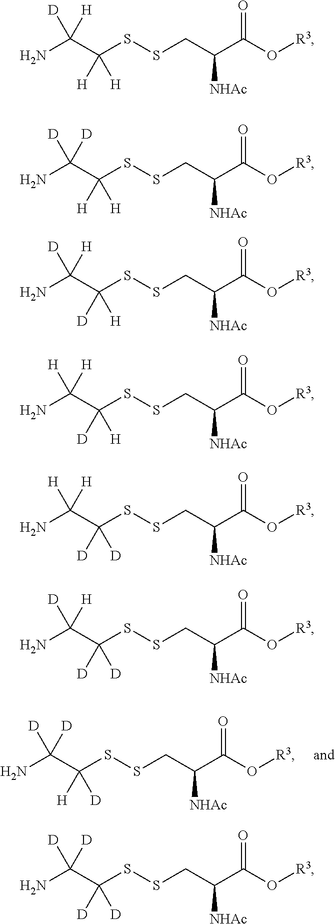
or a pharmaceutically acceptable salt or prodrug thereof, wherein R³ is a (C₁-C₆)alkyl.

22. The compound of claim 20, wherein the pharmaceutically acceptable salt has the structure of Formula III(d): 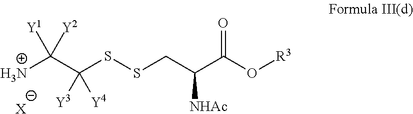
wherein,
Y¹-Y⁴ are each independently selected from H or D;
R³ is a (C₁-C₆)alkyl; and
X is a pharmaceutically acceptable counter ion.

23. The compound of claim 1, wherein the compound has the structure of Formula IV: 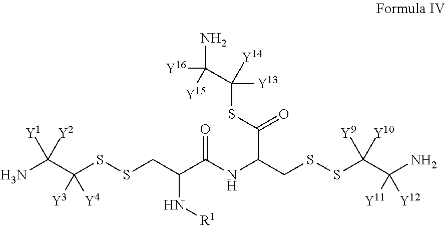
or a pharmaceutically acceptable salt or solvate thereof, wherein:
Y¹-Y⁴ and Y⁹-Y¹⁶ are each independently selected from H or D; and
R¹ is selected from H or an acetyl group.

24. The compound of claim 1, wherein the compound has the structure of Formula V: 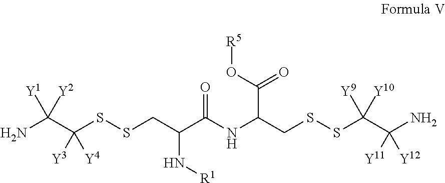
or a pharmaceutically acceptable salt or solvate thereof, wherein:
Y¹-Y⁴ and Y⁹-Y¹⁶ are each independently selected from H or D;
R¹ is selected from H or an acetyl group; and
R⁵ is a (C₁-C₆)alkyl.

25. A compound having the structure of Formula VI: 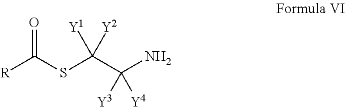
or a pharmaceutically acceptable salt or solvate thereof, wherein:
Y¹-Y⁴ are each independently selected from H or D; and
R is linear or branched aliphatic group (saturated or unsaturated) or aromatic (substituted or non-substituted) having from 1 to 20 carbon atoms.

26. The compound of claim 25, having the structure of Formula VI(a): 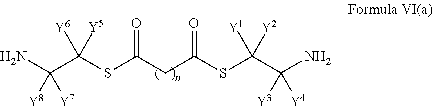
or a pharmaceutically acceptable salt or solvate thereof, wherein:
Y¹-Y⁸ are each independently selected from H or D; and
n is 2-6.

27. The compound of claim 25, wherein the compound is selected from the group consisting of:

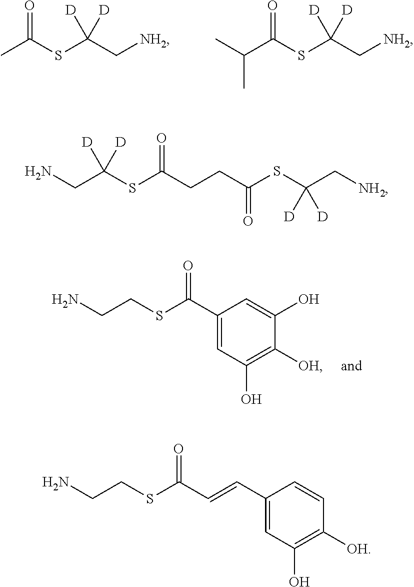

28. A pharmaceutical composition comprising the compound of claim 1 and a pharmaceutically acceptable carrier, diluent, and/or binder.

29-32. (canceled)

33. A method of treating a subject suffering from a disease or disorder selected from the group consisting of cystinosis, fatty liver disease, cirrhosis, an eosinophilic disease or disorder, and Huntington's disease in need of treatment thereof, comprising administering to the subject a therapeutically effective amount of a compound of claim 1.

34-35. (canceled)

36. The method of claim 33, wherein the fatty liver disease is selected from the group consisting of non-alcoholic fatty liver disease (NAFLD), non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy.

37-42. (canceled)

43. A method of reducing fibrosis or fat content or fat accumulation in the liver associated with non-alcoholic fatty liver disease (NAFLD) comprising administering a compound of claim 1.

44. The method of claim 43, wherein the NALFD comprises NASH.

45. A pharmaceutical composition comprising the compound of claim 25 and a pharmaceutically acceptable carrier, diluent, and/or binder.

46. A method of treating a subject suffering from a disease or disorder selected from the group consisting of cystinosis, fatty liver disease, cirrhosis, an eosinophilic disease or disorder, and Huntington's disease in need of treatment thereof, comprising administering to the subject a therapeutically effective amount of a compound of claim 28.

47. A method of reducing fibrosis or fat content or fat accumulation in the liver associated with non-alcoholic fatty liver disease (NAFLD) comprising administering a compound of claim 28.
